(19) United States
(12) Patent Application Publication
(10) Pub. No.: US 2023/0340270 A1
(43) Pub. Date: Oct. 26, 2023

(54) CYANINE COMPOUND AND PHOTOELECTRIC CONVERSION ELEMENT

(71) Applicant: MITSUBISHI GAS CHEMICAL COMPANY, INC., Tokyo (JP)

(72) Inventors: Takashi YAMAMOTO, Tokyo (JP); Kazumasa FUNABIKI, Gifu (JP); Yuta ARISAWA, Gifu (JP); Kenyu AOTANI, Gifu (JP)

(73) Assignee: MITSUBISHI GAS CHEMICAL COMPANY, INC., Tokyo (JP)

(21) Appl. No.: 18/026,687

(22) PCT Filed: Sep. 1, 2021
(86) PCT No.: PCT/JP2021/032131

§ 371 Date: Mar. 16, 2023

(30) Foreign Application Priority Data
Sep. 25, 2020 (JP) 2020-161368

(51) Int. Cl. C09B 23/16; H10K 85/60

(52) U.S. Cl. CPC C09B 23/16; H10K 85/60; H10K 30/20

(57) ABSTRACT

Provided is a cyanine compound being bound counterions consisting of an anion and a cation, wherein the anion is represented by the following formula (I-1):

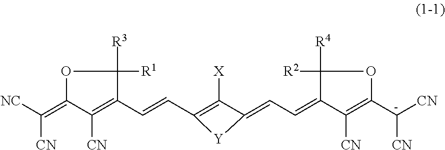

wherein R¹ and R² each independently represent a hydrogen atom or a monovalent organic group; R³ and R⁴ each independently represent a monovalent group such as a phenyl group; X represents a hydrogen atom, a halogen atom or a monovalent organic group; and Y represents a divalent group such as a n-propenyl group.

### TECHNICAL FIELD

The present invention relates to a cyanine compound and a photoelectric conversion element obtained using the same.

### BACKGROUND ART

Techniques of converting visible light to electric signals or electric energy by photoelectric conversion have heretofore been known. The former techniques are widely used in image sensors, and the latter techniques are widely used in solar cells, etc. Also, techniques of photoelectrically converting near-infrared light are used in night vision cameras, various sensors for distance measurement or the like, communication application, analysis apparatuses, etc.

Meanwhile, it is expected that new values can be imparted to the conventional techniques by using elements that are transmissive to visible light, but selectively cause photoelectric conversion of near-infrared light. For example, such a photoelectric conversion element disposed on the foreside of the light-receiving surface of an image sensor enables imaging and sensing (e.g., three-dimensional measurement) to be performed at the same timing using the same element. As a result, the image sensor attains complex functions, downsizing, and cost reduction. Alternatively, such a photoelectric conversion element disposed on the foreside of a display accessorily confers electric energy at the same time with image display outdoors, for example, and can achieve power saving or battery-less approaches.

Laminated organic thin film-type elements are promising as the aforementioned photoelectric conversion elements which selectively cause photoelectric conversion of near-infrared light, in terms of the degree of freedom of design of organic materials, small film thicknesses, and high sensitivity (quantum efficiency). For such a laminated organic thin film-type photoelectric conversion element, it is vital to use, in its photosensitive unit, a material that absorbs only near-infrared light and has no or minimum absorption in the visible light region. Such a near-infrared absorptive material can also be used in optical information recording media, organic solar cells, flash fusing photosensitive materials, thermal shielding films, infrared cut filters, anti-counterfeit ink, or preform heating auxiliary agents intended for plastic bottles, in addition to those mentioned above.

As for examples of the laminated organic thin film-type element which absorbs only near-infrared light and is transmissive to a portion of visible light, for example, Patent Document 1 has reported an example in which a light is selectively absorbed by a material having a local maximum absorption wavelength at 600 to 800 nm using a metal naphthalocyanine derivative. Patent Documents 2 to 4 each describe a photoelectric conversion element having a local maximum absorption wavelength at or around 700 nm in the combined range of visible light and near-infrared light. Particularly, Patent Document 3 states that a material whose absorption intensity at 400 to 550 nm is 1/10 or less of absorption intensity in the near-infrared region is provided. Non Patent Document 1 describes a photoelectric conversion element in which a cyanine color having specific absorption for near-infrared light is used in a photosensitive layer.

### CITATION LIST

### Patent Document

Patent Document 1: Japanese Patent Application Laid-Open No. S63-186251
Patent Document 2: Japanese Patent No. 5270114
Patent Document 3: Japanese Patent Application Laid-Open No. 2012-169676
Patent Document 4: Japanese Patent Application Laid-Open No. 2017-34112

### Non Patent Document

Non Patent Document 1: Org. Lett., Vol. 11, No. 21, 2009

### SUMMARY OF INVENTION

### Technical Problem

However, silicon metal, a typical material for photoelectric conversion elements, also has sensitivity to infrared light at or around a wavelength of 600 to 800 nm, Therefore, the photoelectric conversion elements of Patent Documents 1 to 4 are less likely to demonstrate their superiority over existing techniques. In the case of using a photoelectric conversion element as infrared LED for use in imaging in dark place, three-dimensional distance measurement, etc., or as a light-receiving element for infrared laser light emission, the wavelength of a light emission apparatus is generally a wavelength of 800 nm or more. Hence, use of the elements of Patent Documents 1 to 4 leads to factors for increasing cost, such as the need of using a special light emission apparatus. The material and the photoelectric conversion element of Non Patent Document 5 employ a cyanine color having a maximum wavelength of 800 nm or more, and Non Patent Document 5 describes its quantum efficiency. However, Non Patent Document 5 does not describe the durability performance (e.g., light resistance and heat resistance) of the color, which largely influences the production process of the photoelectric conversion element or the resistance of the element itself. Thus, the degree of feasibility is unknown.

The present invention has been made in light of at least a portion of these circumstances, and an object of the present invention is to provide a novel cyanine compound that more selectively absorbs an incident light of more than 800 nm and is also excellent in light resistance and heat resistance, and a photoelectric conversion element obtained using the cyanine compound.

### Solution to Problem

The present inventors have conducted diligent studies to attain the object and consequently completed the present invention by finding a novel cyanine compound having a local maximum absorption wavelength of more than 800 nm.

Specifically, the present invention is as described below.

[1] A cyanine compound being bound counterions consisting of an anion and a cation, wherein the anion is represented by the following formula (I-1):

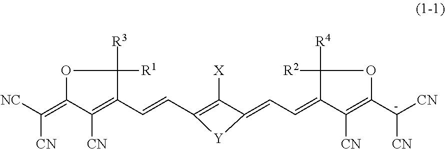

wherein R¹ and R² each independently represent a hydrogen atom or a monovalent organic group; R³ and R⁴ each independently represent a monovalent group represented by the following formula (I-1-1); X represents a hydrogen atom, a halogen atom or a monovalent organic group; and Y represents a divalent group represented by the following formula (I-1-2) or (I-1-3):

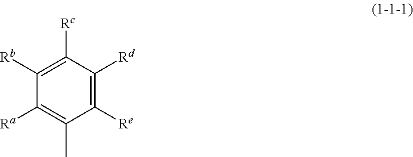

wherein R^{a}, R^{b}, R^{c}, R^{d} and R^{e} each independently represent a hydrogen atom, a monovalent hydrocarbon group or a monovalent electron-withdrawing group; one or more of R^{a}, R^{b}, R^{c}, R^{d} and R^{e} represent the monovalent electron-withdrawing group; and when one of R^{a}, R^{b}, R^{c}, R^{d} and R^{e} is a halogen atom, one or more of the other moieties of R^{a}, R^{b}, R^{c}, R^{d} and R^{e} represent the monovalent hydrocarbon group or the monovalent electron-withdrawing group,

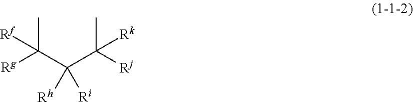

wherein R^{f}, R^{g}, R^{h}, Rⁱ, R^{j} and R^{k} each independently represent a hydrogen atom, or a monovalent hydrocarbon group optionally having oxygen atom(s), nitrogen atom(s) or sulfur atom(s), and

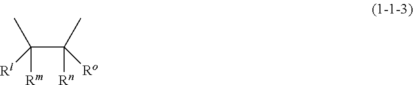

wherein R^{l}, R^{m}, Rⁿ and R^{o} each independently represent a hydrogen atom, or a monovalent hydrocarbon group optionally having oxygen atom(s), nitrogen atom(s) or sulfur atom(s).
[2] The cyanine compound described above, wherein the cation contains one or more selected from the group consisting of an alkali metal cation, an alkaline earth metal cation, an ammonium cation, a sulfonium cation, a phosphonium cation and cationic cyanine.
[3] The cyanine compound described above, wherein the cation contains one or more selected from the group consisting of an alkali metal cation, an ammonium cation and cationic cyanine.
[4] The cyanine compound described above, wherein the cationic cyanine is a cation represented by the following formula (I-2-1), (I-2-2), (I-2-3) or (I-2-4):

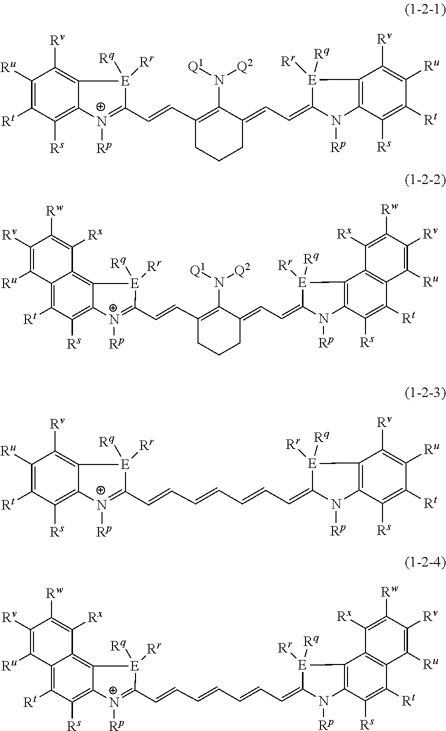

wherein
each E independently represents a carbon atom, a nitrogen atom, an oxygen atom or a sulfur atom;
R^{p}, R^{q}, R^{r}, R^{s}, R^{t}, R^{u}, R^{v}, R^{w} and R^{x} each independently represent a hydrogen atom, a halogen atom, a hydroxy group, a carboxy group, a nitro group, an amino group, an amide group, an imide group, a cyano group, a silyl group, -L¹, —S-L², —SS-L², —SO₂-L³, —N═N-L⁴, or one or more groups selected from the group consisting of groups represented by the following formulas (A), (B), (C), (D), (E), (F), (G) and (H) having one or more combinations of R^{q} and R^{r}, R^{s} and R^{t}, R^{t} and R^{u}, R^{u} and R^{v}, R^{v} and R^{w}, and R^{w} and R^{x} bonded to each other, wherein
the amino group, the amide group, the imide group and the silyl group can be each further substituted by one or more groups L selected from the group consisting of a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms and a monovalent heterocyclic group having 3 to 14 carbon atoms;
each of the L¹ and the L⁴ is a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L;
the L² is a hydrogen atom, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L; and
the L³ is a hydroxy group, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L;
Q¹ represents an acetyl group; and Q² represents a structure represented by the following formula (q1), (q2) or (q3):

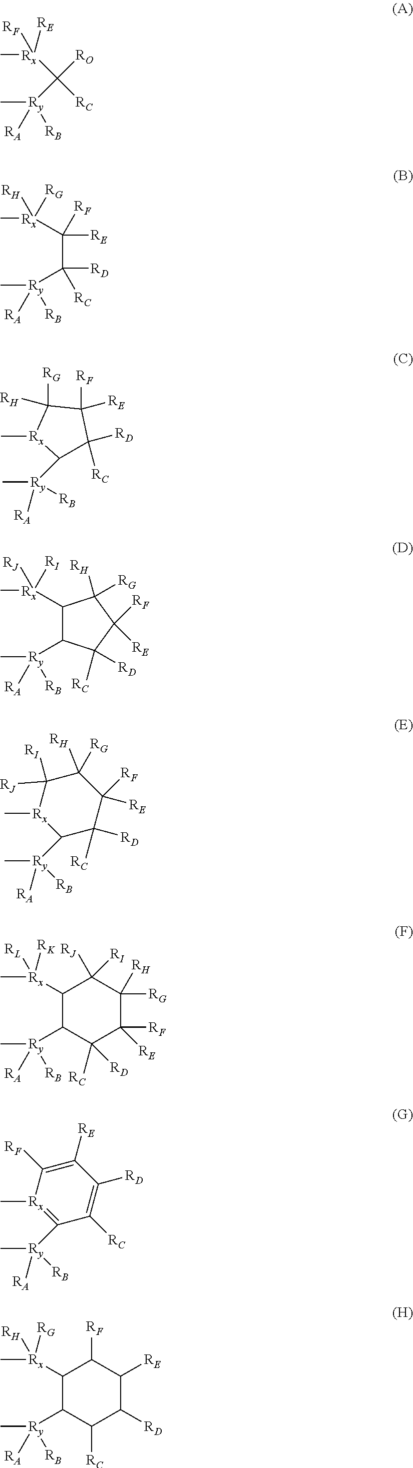

wherein the combination of R^{x} and R^{y} is a combination of R^{q} and R^{r}, R^{s} and R^{t}, R^{t} and R^{u}, R^{u} and R^{v}, R^{v} and R^{w}, or R^{w} and R^{x};
R_{A}, R_{B}, R_{C}, R_{D}, R_{E}, R_{F}, R_{G}, R_{H}, R_{I}, R_{J}, R_{K} and R_{L} each independently represent a hydrogen atom, a halogen atom, a hydroxy group, a carboxy group, a nitro group, an amino group, an amide group, an imide group, a cyano group, a silyl group, -L¹, —S-L², —SS-L², —SO₂-L³ or —N═N-L⁴, wherein L¹, L², L³ and L⁴ are as defined in L¹, L², L³ and L⁴ in the formulas (I-2-1) and (I-2-2), and the amino group, the amide group, the imide group and the silyl group can be substituted by the group(s) L,

—CₘHₘ₊₁ (q1)

—CₐHₐ₊₁—OC_{b}H_{b+1} (q2)

wherein m in the formula (q1) represents an integer of 1 to 5, and a and b in the formula (q2) each represent an integer of 1 to 5, and

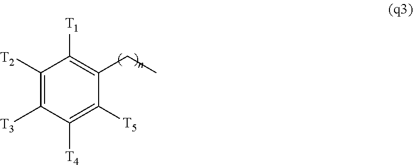

wherein n represents an integer of 1 to 5; T₁, T₂, T₃, T₄ and T₅ each independently represent a hydrogen atom or —OCₚHₚ₊₁; and p represents an integer of 1 to 5.
[5] The cyanine compound described above, wherein the monovalent organic group represented by each of the R¹ and the R² is a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by a monovalent hydrocarbon group or a monovalent electron-withdrawing group.
[6] The cyanine compound described above, wherein the R¹ and the R² are each independently a hydrogen atom, a monovalent aliphatic hydrocarbon group having 1 to 3 carbon atoms or a monovalent group represented by the formula (I-1-1).
[7] The cyanine compound described above, wherein
the monovalent organic group represented by the X represents a hydroxy group, a carboxy group, a nitro group, an amino group, an amide group, an imide group, a cyano group, a silyl group, -L¹, —S-L², —SS-L³, —SO₂-L³, or —N═N-L⁴, wherein
the amino group, the amide group, the imide group and the silyl group can be each further substituted by one or more groups L selected from the group consisting of a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms and a monovalent heterocyclic group having 3 to 14 carbon atoms;
each of the L¹ and the L⁴ is a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L;
the L² is a hydrogen atom, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L; and
the L³ is a hydroxy group, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L.
   [8] The cyanine compound described above, wherein the X is a halogen atom.
   [9] The cyanine compound described above, wherein the monovalent hydrocarbon group represented by each of the R^{a}, the R^{b}, the R^{c}, the R^{d} and the R^{e} is a monovalent aliphatic hydrocarbon group having 1 to 12 carbon atoms, a monovalent alicyclic hydrocarbon group having 3 to 14 carbon atoms or a monovalent aromatic hydrocarbon group having 6 to 14 carbon atoms, each of which can be further substituted by one or more groups selected from the group consisting of a monovalent aliphatic hydrocarbon group having 1 to 12 carbon atoms, a monovalent alicyclic hydrocarbon group having 3 to 14 carbon atoms and a monovalent aromatic hydrocarbon group having 6 to 14 carbon atoms.
   [10] The cyanine compound described above, wherein the monovalent electron-withdrawing group represented by each of the R^{a}, the R^{b}, the R^{c}, the R^{d} and the R^{e} is a halogen atom, a carboxy group, a nitro group, a cyano group, a group represented by —COR, a group represented by —CONR₂, a group represented by —SO₂R or a group represented by —SO₃R, wherein the R is as defined in the monovalent hydrocarbon group or a hydrogen atom.
   [11] The cyanine compound described above, wherein the R^{a}, the R^{b}, the R^{c}, the R^{d} and the R^{e} each independently represent a hydrogen atom or a halogen atom, and two or more of R^{a}, R^{b}, R^{c}, R^{d} and R^{e} are halogen atoms.
   [12] The cyanine compound described above, wherein the monovalent hydrocarbon group optionally having oxygen atom(s), nitrogen atom(s) or sulfur atom(s), represented by each of the R^{f}, the R^{g}, the R^{h}, the Rⁱ, the R^{j}, the R^{k}, the R^{l}, the R^{m}, the Rⁿ and the R^{o} is a monovalent aliphatic hydrocarbon group having 1 to 12 carbon atoms, a monovalent alicyclic hydrocarbon group having 3 to 14 carbon atoms or a monovalent aromatic hydrocarbon group having 6 to 14 carbon atoms, each of which can be further substituted by one or more groups selected from the group consisting of a monovalent aliphatic hydrocarbon group having 1 to 12 carbon atoms, a monovalent alicyclic hydrocarbon group having 3 to 14 carbon atoms and a monovalent aromatic hydrocarbon group having 6 to 14 carbon atoms, each of which optionally has oxygen atom(s), nitrogen atom(s) or sulfur atom(s).
   [13] The cyanine compound described above, wherein R^{f}, R^{g}, R^{h}, Rⁱ, R^{j}, R^{k}, R^{l}, R^{m}, Rⁿ and R^{o} each independently represent a hydrogen atom, or a monovalent aliphatic hydrocarbon group having 1 to 12 carbon atoms.
   [14] A photoelectric conversion element including an infrared photoelectric conversion unit including a pair of electrodes and an organic infrared photoelectric conversion film disposed between the pair of electrodes, wherein
the organic infrared photoelectric conversion film contains the cyanine compound described above.
   [15] The photoelectric conversion element described above, wherein the organic infrared photoelectric conversion film contains an organic n-type semiconductor and/or an organic p-type semiconductor.
   [16] The photoelectric conversion element described above, wherein the infrared photoelectric conversion unit includes one or more selected from the group consisting of a hole transport layer, an electron transport layer, a hole blocking layer, and an electron blocking layer between the electrode and the organic infrared photoelectric conversion film.
   [17] The photoelectric conversion element described above, wherein in the infrared photoelectric conversion unit, a local maximum absorption wavelength and a maximum absorption wavelength of optical absorption spectra in the infrared region are 800 nm or more and 2500 nm or less.
   [18] The photoelectric conversion element described above, wherein the photoelectric conversion element further includes a visible photoelectric conversion unit having sensitivity to a light in the visible region.

### Advantageous Effects of Invention

The present invention can provide a cyanine compound that more selectively absorbs an incident light of more than 800 nm and is also excellent in light resistance and heat resistance, and a photoelectric conversion element obtained using the cyanine compound.

### BRIEF DESCRIPTION OF DRAWING

FIG. 1 is a cross-sectional schematic view partially showing one example of the photoelectric conversion unit of the present invention.

FIG. 2 shows absorption spectra as to one example of the cyanine compound of the present invention.

### DESCRIPTION OF EMBODIMENTS

Hereinafter, the mode for carrying out the present invention (hereinafter, simply referred to as the “present embodiment”) will be described in detail with reference to the drawings, if necessary. However, the present invention is not limited by the present embodiment described below. Various changes or modifications can be made in the present invention without departing from the spirit of the present invention. In the drawings, the same numerals or symbols will be used to designate the same components, so that the description will be omitted. Positional relationship indicated by terms such as “up”, “down”, “right” and “left” is based on the positional relationship shown in the drawings, unless otherwise specified. The dimensional ratios of the drawings are not limited to the shown ratios.

(Cyanine Compound)

The cyanine compound of the present embodiment is a cyanine compound being bound counterions consisting of an anion and a cation, wherein the anion is represented by the following formula (I-1):

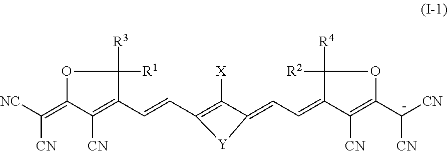

In the formula (I-1), R¹ and R² each independently represent a hydrogen atom or a monovalent organic group; R³ and R⁴ each independently represent a monovalent group represented by the following formula (I-1-1); X represents a hydrogen atom, a halogen atom or a monovalent organic group; and Y represents a divalent group represented by the following formula (I-1-2) or (I-1-3):

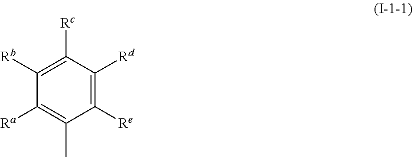

In the formula (I-1-1), R^{a}, R^{b}, R^{c}, R^{d} and R^{e} each independently represent a hydrogen atom, a monovalent hydrocarbon group or a monovalent electron-withdrawing group; one or more of R^{a}, R^{b}, R^{c}, R^{d} and R^{e} represent the monovalent electron-withdrawing group; and when one of R^{a}, R^{b}, R^{c}, R^{d} and R^{e} is a halogen atom, one or more of the other moieties of R^{a}, R^{b}, R^{c}, R^{d} and R^{e} represent the monovalent hydrocarbon group or the monovalent electron-withdrawing group.

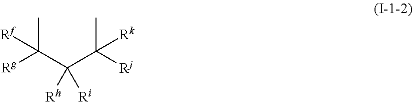

In the formula (I-1-2), R^{f}, R^{g}, R^{h}, Rⁱ, R^{j} and R^{k} each independently represent a hydrogen atom, or a monovalent hydrocarbon group optionally having oxygen atom(s), nitrogen atom(s) or sulfur atom(s).

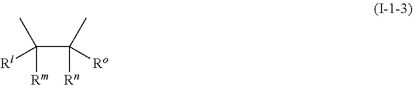

In the formula (I-1-3), R^{l}, R^{m}, Rⁿ and R^{o} each independently represent a hydrogen atom, or a monovalent hydrocarbon group optionally having oxygen atom(s), nitrogen atom(s) or sulfur atom(s).

(Anion)

The anion according to the present embodiment is represented by the formula (I-1). Each of R¹, R², R³, R⁴, X and Y preferably has a total of 60 or less carbon atoms, more preferably 50 or less carbon atoms, particularly preferably 40 or less carbon atoms, including substituent(s). When the number of carbon atoms falls within this range, the cyanine compound is more easily synthesized, while absorption intensity per unit weight tends to be high.

Examples of the monovalent organic group represented by each of R¹ and R² include, but are not particularly limited, a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by a monovalent hydrocarbon group or a monovalent electron-withdrawing group.

Examples of the monovalent aliphatic hydrocarbon group having 1 to 12 carbon atoms include: alkyl groups such as a methyl group (Me), an ethyl group (Et), a n-propyl group (n-Pr), an isopropyl group (i-Pr), a n-butyl group (n-Bu), a sec-butyl group (s-Bu), a tert-butyl group (t-Bu), a pentyl group, a hexyl group, an octyl group, a nonyl group, a decyl group and a dodecyl group; alkenyl groups such as a vinyl group, a 1-propenyl group, a 2-propenyl group, a butenyl group, a 1,3-butadienyl group, a 2-methyl-1-propenyl group, a 2-pentenyl group, a hexenyl group and an octenyl group; and alkynyl groups such as an ethynyl group, a propynyl group, a butynyl group, a 2-methyl-1-propynyl group, a hexynyl group and an octynyl group. Among them, a monovalent aliphatic hydrocarbon group having 1 to 3 carbon atoms is preferred. Specifically, an alkyl group having 1 to 3 carbon atoms, such as a methyl group (Me), an ethyl group (Et), a n-propyl group (n-Pr) and an isopropyl group (i-Pr) is preferred.

Examples of the monovalent halogen-substituted alkyl group having 1 to 12 carbon atoms include monovalent halogen-substituted alkyl groups having 1 to 3 carbon atoms. More specific examples of such a halogen-substituted alkyl group include a trichloromethyl group, a trifluoromethyl group, a 1,1-dichloroethyl group, a pentachloroethyl group, a pentafluoroethyl group, a heptachloropropyl group and a heptafluoropropyl group.

Examples of the monovalent alicyclic hydrocarbon group having 3 to 14 carbon atoms include monovalent alicyclic hydrocarbon groups having 4 to 10 carbon atoms. More specific examples of such an alicyclic hydrocarbon group include: cycloalkyl groups such as a cyclobutyl group, a cyclopentyl group, a cyclohexyl group, a cycloheptyl group and a cyclooctyl group; and polycyclic alicyclic groups such as a norbornane group and an adamantane group.

Examples of the monovalent aromatic hydrocarbon group having 6 to 14 carbon atoms include a phenyl group, a tolyl group, a xylyl group, a mesityl group, a cumenyl group, a 1-naphthyl group, a 2-naphthyl group, an anthracenyl group, a phenanthryl group, an acenaphthyl group, a phenalenyl group, a tetrahydronaphthyl group, an indanyl group and a biphenylyl group. This aromatic hydrocarbon group may be a monovalent group represented by the formula (I-1-1) mentioned later in detail. In this case, R¹ and R³ or R² and R⁴ may be the same with or different from each other.

Examples of the heterocyclic group having 3 to 14 carbon atoms include groups consisting of heterocyclic rings such as furan, thiophene, pyrrole, pyrazole, imidazole, triazole, oxazole, oxadiazole, thiazole, thiadiazole, indole, indoline, indolenine, benzofuran, benzothiophene, carbazole, dibenzofuran, dibenzothiophene, pyridine, pyrimidine, pyrazine, pyridazine, quinoline, isoquinoline, acridine, morpholine and phenazine.

Examples of the monovalent hydrocarbon group serving as a substituent include, but are not particularly limited to, monovalent aliphatic hydrocarbon groups having 1 to 12 carbon atoms, monovalent alicyclic hydrocarbon groups having 3 to 14 carbon atoms, and monovalent aromatic hydrocarbon groups having 6 to 14 carbon atoms. Each of their examples or preferred forms is the same as those described above, so that the description will be omitted here.

The monovalent electron-withdrawing group serving as a substituent is not particularly limited as long as it is a monovalent group that exhibits electron-withdrawing properties in the anion according to the present embodiment. In this context, whether or not the substituent is an “electron-withdrawing group” can be determined as follows: an anion molecule having the substituent is structurally optimized by molecular simulation using density functional formalism (e.g., molecular simulation using quantum chemical calculation program Gaussian manufactured by Gaussian, Inc.) to determine electron affinity or ionization energy. This is referred to as pre-substitution electron affinity or ionization energy. Subsequently, electron affinity or ionization energy is determined in the same manner as above as to an anion molecule obtained by substituting the substituent in the anion molecule by a hydrogen atom or a monovalent hydrocarbon group. This is referred to as post-substitution electron affinity or ionization energy. When the post-substitution electron affinity or ionization energy is larger than the pre-substitution electron affinity or ionization energy, this substituent is determined as an electron-withdrawing group. Such a monovalent electron-withdrawing group will be mentioned later in detail, so that the description will be omitted here.

In the monovalent group represented by the formula (I-1-1), R^{a}, R^{b}, R^{c}, R^{d} and R^{e} (hereinafter, simply referred to as “R^{a} to R^{e}”) each independently represent a hydrogen atom, a monovalent hydrocarbon group or a monovalent electron-withdrawing group. One or more of R^{a} to R^{e} represent the monovalent electron-withdrawing group, i.e., the monovalent group represented by the formula (I-1-1) inevitably has a monovalent electron-withdrawing group. When one of R^{a} to R^{e} is a halogen atom, one or more of the other moieties of R^{a} to R^{e} represent the monovalent hydrocarbon group or the monovalent electron-withdrawing group. Specifically, when one of R^{a} to R^{e} is a halogen atom, there is no form in which all the other moieties of R^{a} to R^{e} are hydrogen atoms. Examples of the monovalent hydrocarbon group include the same as those listed above, so that the description will be omitted here.

The monovalent electron-withdrawing group is not particularly limited as long as it is a monovalent group that exhibits electron-withdrawing properties in the anion according to the present embodiment. Examples of such a monovalent electron-withdrawing group include a halogen atom, a carboxy group (—COOH), a nitro group (—NO₂), a cyano group (—CN), a group represented by —COR, a group represented by —CONR₂, a group represented by —SO₂R or a group represented by —SO₃R. In this context, R is a hydrogen atom or a monovalent hydrocarbon group. The monovalent hydrocarbon group is as defined in the monovalent hydrocarbon group described above, so that the detailed description will be omitted here.

Examples of the halogen atom include a fluorine atom (F), a chlorine atom (Cl), a bromine atom (Br) and an iodine atom (I).

Examples of the group represented by —COR (acyl group) include an acetyl group, a propionyl group, a butyryl group, an isobutyryl group, a benzoyl group, an acryloyl group and a methacryloyl group. In this context, the number of carbon atoms in R may be 1 to 6.

Examples of the group represented by —CONR₂ (amide group) include an amide group, a methylamide group, a dimethylamide group, a diethylamide group, a dipropylamide group, a diisopropylamide group, and a dibutylamide group. In this context, the group represented by —CONR₂ may be lactam in which one of the R moieties is bonded to a carbon atom of a carboxy group. Examples of the lactam include an α-lactam group, a β-lactam group, a γ-lactam group, and a δ-lactam group. In this context, the number of carbon atoms in R may be 1 to 4.

Examples of the group represented by —SO₂R include a mesyl group, an ethylsulfonyl group, a n-butylsulfonyl group, a phenylsulfonyl group and a p-toluenesulfonyl group. In this context, the number of carbon atoms in R may be 1 to 7.

Examples of the group represented by —SO₃R include a sulfo group (—SO₃H), a methylsulfonic acid group (—SO₃CH₃), an ethylsulfonic acid group (—SO₃C₂H₅), a n-butylsulfonic acid group (—SO₃C₃H₇), and a phenylsulfonic acid group (—SO₃C₆H₅). In this context, the number of carbon atoms in R may be 1 to 6.

In the present embodiment, preferably two or more of R^{a} to R^{e} in the anion are electron-withdrawing groups, more preferably three or more thereof are electron-withdrawing groups, and particularly preferably all of them are electron-withdrawing groups, from the viewpoint of still more selectively absorbing an incident light of more than 800 nm, From a similar viewpoint, the electron-withdrawing group is preferably a halogen atom. When two or more of R^{a} to R^{e} are electron-withdrawing groups, more preferably all of them are halogen atoms.

The combination of R^{a} to R^{e} in the formula (I-1-1) may be any combination of the substituents listed above as long as it is a combination in which, when one or more of R^{a} to R^{e} represent the monovalent electron-withdrawing group and one of R^{a} to R^{e} is a halogen atom, one or more of the other moieties of R^{a} to R^{e} represent the monovalent hydrocarbon group or the monovalent electron-withdrawing group.

X represents a hydrogen atom, a halogen atom or a monovalent organic group. Among them, a halogen atom is preferred. Examples of the halogen atom include the same as those listed above, so that the description will be omitted here.

Examples of the monovalent organic group represented by X include, but are not particularly limited, a hydroxy group, a carboxy group, a nitro group, an amino group, an amide group, an imide group, a cyano group, a silyl group, -L¹, —S-L², —SS-L³, —SO₂-L³, or —N═N-L⁴. The amino group, the amide group, the imide group and the silyl group can be each further substituted by one or more groups L selected from the group consisting of a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms and a monovalent heterocyclic group having 3 to 14 carbon atoms.

Each of L¹ and L⁴ is a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L. L² is a hydrogen atom, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L. L³ is a hydroxy group, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a monovalent heterocyclic group having 3 to 14 carbon atoms, each of which can be further substituted by the group(s) L.

Examples of the monovalent aliphatic hydrocarbon group having 1 to 12 carbon atoms, the monovalent halogen-substituted alkyl group having 1 to 12 carbon atoms, the monovalent alicyclic hydrocarbon group having 3 to 14 carbon atoms, the monovalent aromatic hydrocarbon group having 6 to 14 carbon atoms and the monovalent heterocyclic group having 3 to 14 carbon atoms include the same as those listed above, so that the description will be omitted here.

The monovalent aliphatic hydrocarbon group having 1 to 12 carbon atoms which can be further substituted by the group(s) L is preferably a methyl group, an ethyl group, a n-propyl group, an isopropyl group, a n-butyl group, a sec-butyl group, a tert-butyl group, a pentyl group, a hexyl group, an octyl group, a 4-phenylbutyl group, or a 2-cyclohexylethyl, more preferably a methyl group, an ethyl group, a n-propyl group, an isopropyl group, a n-butyl group, a sec-butyl group, a tert-butyl group, or a hexyl group.

The monovalent halogen-substituted alkyl group having 1 to 12 carbon atoms which can be further substituted by the group(s) L is preferably a trichloromethyl group, a pentachloroethyl group, a trifluoromethyl group, a pentafluoroethyl group, or a 5-cyclohexyl-2,2,3,3-tetrafluoropentyl group, more preferably a trichloromethyl group, a pentachloroethyl group, a trifluoromethyl group, or a pentafluoroethyl group.

The monovalent alicyclic hydrocarbon group having 3 to 14 carbon atoms which can be further substituted by the group(s) L is preferably a cyclobutyl group, a cyclopentyl group, a cyclohexyl group, a 4-ethylcyclohexyl group, a cyclooctyl group, or a 4-phenylcycloheptyl group, more preferably a cyclopentyl group, a cyclohexyl group, or a 4-ethylcyclohexyl group.

The monovalent aromatic hydrocarbon group having 6 to 14 carbon atoms which can be further substituted by the group(s) L is preferably a phenyl group, a 1-naphthyl group, a 2-naphthyl group, a tolyl group, a xylyl group, a mesityl group, a cumenyl group, a 3,5-di-tert-butylphenyl group, a 4-cyclopentylphenyl group, a 2,3,6-triphenylphenyl group, or a 2,3,4,5,6-pentaphenylphenyl group, more preferably a phenyl group, a tolyl group, a xylyl group, a mesityl group, a cumenyl group, or a 2,3,4,5,6-pentaphenylphenyl group.

The monovalent heterocyclic group having 3 to 14 carbon atoms which can be further substituted by the group(s) L is preferably a group consisting of furan, thiophene, pyrrole, indole, indoline, indolenine, benzofuran, benzothiophene, or morpholine, more preferably a group consisting of furan, thiophene, pyrrole, or morpholine.

The monovalent aliphatic hydrocarbon group having 1 to 12 carbon atoms, the monovalent halogen-substituted alkyl group having 1 to 12 carbon atoms, the monovalent alicyclic hydrocarbon group having 3 to 14 carbon atoms, the monovalent aromatic hydrocarbon group having 6 to 14 carbon atoms or the heterocyclic group having 3 to 14 carbon atoms, each of which can be further substituted by the group(s) L, may further have one or more selected from the group consisting of a halogen atom, a sulfo group, a hydroxy group, a cyano group, a nitro group, a carboxy group, a phosphoric acid group and an amino group. Examples thereof include a 4-sulfobutyl group, a 4-cyanobutyl group, a 5-carboxypentyl group, a 5-aminopentyl group, a 3-hydroxypropyl group, a 2-phosphorylethyl group, a 6-amino-2,2-dichlorohexyl group, a 2-chloro-4-hydroxybutyl group, a 2-cyanocyclobutyl group, a 3-hydroxycyclopentyl group, a 3-carboxycyclopentyl group, a 4-aminocyclohexyl group, a 4-hydroxycyclohexyl group, a 4-hydroxyphenyl group, a 2-hydroxynaphthyl group, a 4-aminophenyl group, a 2,3,4,5,6-pentafluorophenyl group, a 4-nitrophenyl group, a group consisting of 3-methylpyrrole, a 2-hydroxyethoxy group, a 3-cyanopropoxy group, a 4-fluorobenzoyl group, a 2-hydroxyethoxycarbonyl group, and a 4-cyanobutoxycarbonyl group.

Examples of the amino group optionally having the group(s) L include an amino group, an ethylamino group, a dimethylamino group, a methylethylamino group, a dibutylamino group, and a diisopropylamino group.

Examples of the amide group optionally having the group(s) L include an amide group, a methylamide group, a dimethylamide group, a diethylamide group, a dipropylamide group, a diisopropylamide group, a dibutylamide group, an α-lactam group, a β-lactam group, a γ-lactam group, and a δ-lactam group.

Examples of the imide group optionally having the group(s) L include an imide group, a methylimide group, an ethylimide group, a diethylimide group, a dipropylimide group, a diisopropylimide group, and a dibutylimide group.

Examples of the silyl group optionally having the group(s) L include a trimethylsilyl group, a tert-butyldimethylsilyl group, a triphenylsilyl group, and a triethylsilyl group.

Y represents a divalent group represented by the formula (I-1-2) or (I-1-3). In the formula (I-1-2), R^{f}, R^{g}, R^{h}, Rⁱ, R^{j} and R^{k} (hereinafter, simply referred to as “R^{f} to R^{k}”) each independently represent a hydrogen atom, or a monovalent hydrocarbon group optionally having oxygen atom(s), nitrogen atom(S) or sulfur atom(s). In the formula (I-1-3), R^{l}, R^{m}, Rⁿ and R^{o}(hereinafter, simply referred to as “R^{l} to R^{o}”) each independently represent a hydrogen atom, or a monovalent hydrocarbon group optionally having oxygen atom(s), nitrogen atom(s) or sulfur atom(s).

Examples of the monovalent hydrocarbon group optionally having oxygen atom(s), nitrogen atom(s) or sulfur atom(s) include a monovalent aliphatic hydrocarbon group having 1 to 12 carbon atoms, a monovalent alicyclic hydrocarbon group having 3 to 14 carbon atoms or a monovalent aromatic hydrocarbon group having 6 to 14 carbon atoms, each of which can be further substituted by one or more groups selected from the group consisting of a monovalent aliphatic hydrocarbon group having 1 to 12 carbon atoms, a monovalent alicyclic hydrocarbon group having 3 to 14 carbon atoms and a monovalent aromatic hydrocarbon group having 6 to 14 carbon atoms, each of which optionally has oxygen atom(s), nitrogen atom(s) or sulfur atom(s).

Examples of the monovalent hydrocarbon group without having an oxygen atom, a nitrogen atom and a sulfur atom, more specifically, the monovalent aliphatic hydrocarbon group having 1 to 12 carbon atoms, the monovalent alicyclic hydrocarbon group having 3 to 14 carbon atoms and the monovalent aromatic hydrocarbon group having 6 to 14 carbon atoms, as well as the monovalent aliphatic hydrocarbon group having 1 to 12 carbon atoms, the monovalent alicyclic hydrocarbon group having 3 to 14 carbon atoms and the monovalent aromatic hydrocarbon group having 6 to 14 carbon atoms, each serving as a substituent include the same as those listed about the monovalent hydrocarbon group described above, so that the description will be omitted here.

Examples of the monovalent hydrocarbon group having an oxygen atom, a nitrogen atom or a sulfur atom include a monovalent aliphatic hydrocarbon group having 1 to 12 carbon atoms, a monovalent alicyclic hydrocarbon group having 3 to 14 carbon atoms or a monovalent aromatic hydrocarbon group having 6 to 14 carbon atoms, each of which can be further substituted by one or more groups selected from the group consisting of a monovalent aliphatic hydrocarbon group having 1 to 12 carbon atoms, a monovalent alicyclic hydrocarbon group having 3 to 14 carbon atoms and a monovalent aromatic hydrocarbon group having 6 to 14 carbon atoms (hereinafter, simply referred to as “substituent(s)” in the description of Y), each of which has one or more selected from the group consisting of an oxygen atom, a nitrogen atom and a sulfur atom. Examples of the case of having an oxygen atom include the case of having a hydroxy group, an ether group, a carbonyl group or a carboxy group. Examples of the case of having a nitrogen atom include the case of having a cyano group or an amino group. Examples of the case of having a sulfur atom include the case of having a thioether group. Examples of the case of having an oxygen atom and a nitrogen atom include the case of having a nitro group. Examples of the case of having an oxygen atom and a sulfur atom include the case of having a sulfo group.

The hydrogen atom, or the monovalent hydrocarbon group optionally having oxygen atom(s), nitrogen atom(s) or sulfur atom(s) is preferably a hydrogen atom, or a monovalent aliphatic hydrocarbon group having 1 to 12 carbon atoms. The monovalent aliphatic hydrocarbon group having 1 to 12 carbon atoms is more preferably a monovalent aliphatic hydrocarbon group having 1 to 6 carbon atoms, further preferably a monovalent aliphatic hydrocarbon group having 1 to 4 carbon atoms.

Preferred examples of the combinations of R^{f} to R^{k} and R^{l} to R^{o} include combinations shown in the following table.

TABLE 1

| R^{f} | R^{g} | R^{h} | Rⁱ | R^{j} | R^{k} |
|---|---|---|---|---|---|
| H | H | H | H | H | H |
| H | H | t-Bu | H | H | H |
| H | H | Me | H | H | H |
| H | H | Me | Me | H | H |
| H | H | Et | H | H | H |

| R^{l} | R^{m} | Rⁿ | R^{o} |
|---|---|---|---|
| H | H | H | H |
| H | Me | H | Me |

(Cation)

The cation according to the present embodiment is not particularly limited and preferably contains one or more selected from the group consisting of an alkali metal cation, an alkaline earth metal cation, an ammonium cation, a sulfonium cation, a phosphonium cation and cationic cyanine. The cation more preferably contains one or more selected from the group consisting of an alkali metal cation, an ammonium cation and cationic cyanine.

Examples of the alkali metal cation include a lithium cation (Li⁺), a sodium cation (Na⁺), a potassium cation (K⁺), a rubidium cation (Rb⁺) and a cesium cation (Cs⁺).

Examples of the alkaline earth metal cation include a beryllium cation (Be²⁺), a magnesium cation (Mg²⁺), a calcium cation (Ca²⁺), a strontium cation (Sr²⁺) and a barium cation (Ba²⁺).

The ammonium cation includes an ammonium ion (NH₄⁺), primary ammonium cations (NH₃R⁺), secondary ammonium cations (NH₂R₂⁺), tertiary ammonium cations (NHR₃⁺) and quaternary ammonium cations (HR₄⁺) such as tetraalkylammonium cations typified by a tetrabutylammonium cation. In this context, R represents a monovalent aliphatic hydrocarbon group having 1 to 12 carbon atoms, such as an alkyl group having 1 to 12 carbon atoms, a monovalent alicyclic hydrocarbon group having 3 to 14 carbon atoms, or a monovalent aromatic hydrocarbon group having 6 to 14 carbon atoms.

The sulfonium cation includes a sulfonium ion (SH₃⁺), primary sulfonium cations (SH₂R⁺), secondary sulfonium cations (SHR₂⁺) and tertiary sulfonium cations (SR₃⁺). In this context, R represents a monovalent aliphatic hydrocarbon group having 1 to 12 carbon atoms, such as an alkyl group having 1 to 12 carbon atoms, a monovalent alicyclic hydrocarbon group having 3 to 14 carbon atoms, or a monovalent aromatic hydrocarbon group having 6 to 14 carbon atoms.

The phosphonium cation includes a phosphonium ion (PH₄⁺), primary phosphonium cations (PH₃R⁺), secondary phosphonium cations (PH₂R₂⁺), tertiary phosphonium cations (PHR₃⁺) and quaternary phosphonium cations (PR₄⁺). In this context, R represents a monovalent aliphatic hydrocarbon group having 1 to 12 carbon atoms, such as an alkyl group having 1 to 12 carbon atoms, a monovalent alicyclic hydrocarbon group having 3 to 14 carbon atoms, or a monovalent aromatic hydrocarbon group having 6 to 14 carbon atoms.

Examples of the cationic cyanine include cations represented by the following formula (I-2-1), (I-2-2), (I-2-3) or (I-2-4):

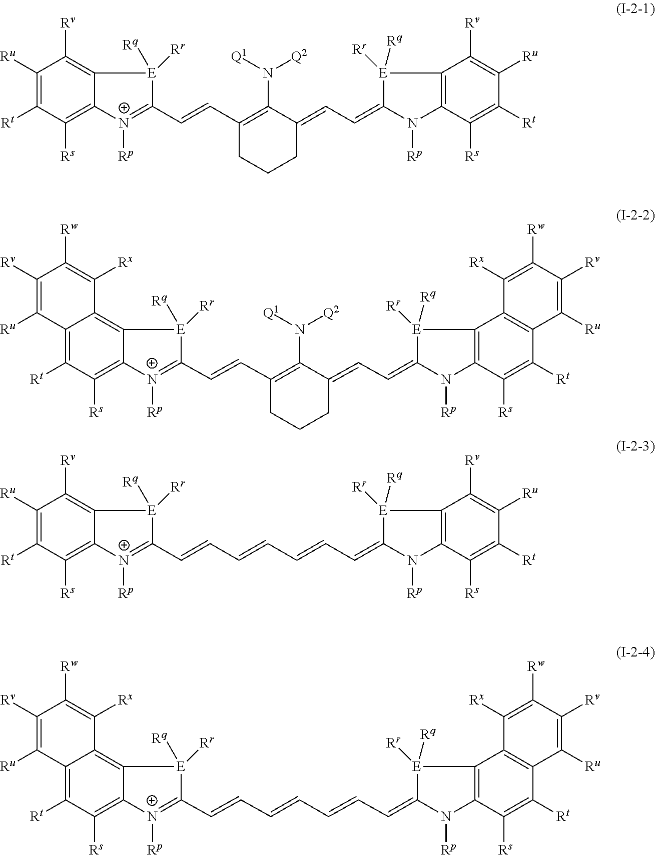

In the formulas (I-2-1), (I-2-2), (I-2-3) and (I-2-4), each E independently represents a carbon atom, a nitrogen atom, an oxygen atom or a sulfur atom, R^{p}, R^{q}, R^{r}, R^{s}, R^{t}, R^{u}, R^{v}, R^{w} and R^{x} each independently represent a hydrogen atom, a halogen atom, a hydroxy group, a carboxy group, a nitro group, an amino group, an amide group, an imide group, a cyano group, a silyl group, -L¹, —S-L², —SS-L², —SO₂-L³, —N═N-L⁴, or one or more groups selected from the group consisting of groups represented by the following formulas (A), (B), (C), (D), (E), (F), (G) and (H) having one or more combinations of R^{q} and R^{r}, R^{s} and R^{t}, R^{t} and R^{u}, R^{u} and R^{v}, R^{v} and R^{w}, and R^{w} and R^{x} bonded to each other.

The amino group, the amide group, the imide group and the silyl group can be each further substituted by one or more groups L selected from the group consisting of a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms and a monovalent heterocyclic group having 3 to 14 carbon atoms.

Each of L¹ and L⁴ is a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L.

L² is a hydrogen atom, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L.

L³ is a hydroxy group, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L.

Q¹ represents an acetyl group, and Q² represents a structure represented by the following formula (q1), (q2) or (q3):

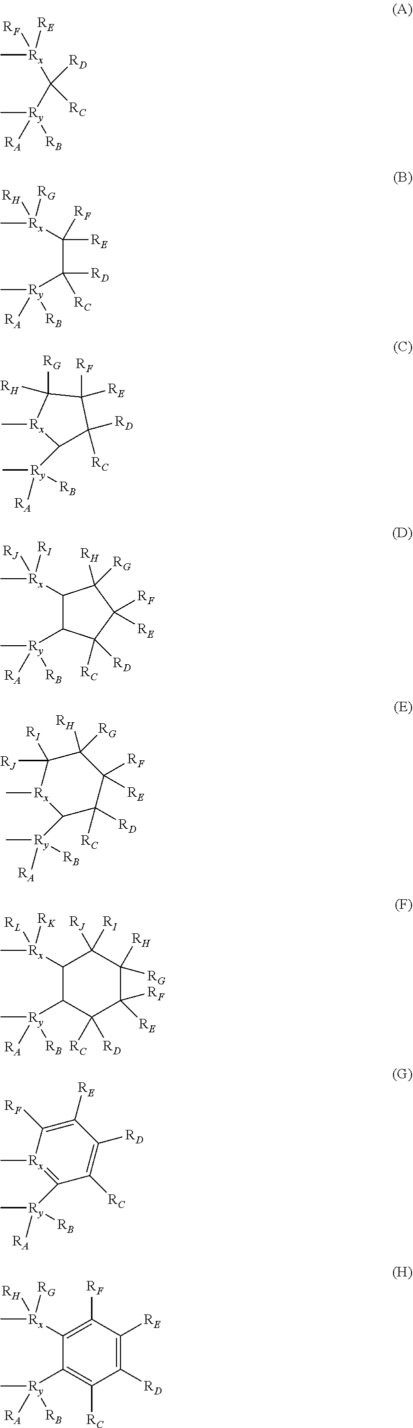

In the formulas (A), (B), (C), (D), (E), (F), (G) and (H), the combination of R^{x} and R^{y} is a combination of R^{q} and R^{r}, R^{s} and R^{t}, R^{t} and R^{u}, R^{u} and R^{v}, R^{v} and R^{w}, or R^{w} and R^{x}.

R_{A}, R_{B}, R_{C}, R_{D}, R_{E}, R_{F}, R_{G}, R_{H}, R_{I}, R_{J}, R_{K} and R_{L} each independently represent a hydrogen atom, a halogen atom, a hydroxy group, a carboxy group, a nitro group, an amino group, an amide group, an imide group, a cyano group, a silyl group, -L¹, —S-L², —SS-L², —SO₂-L³ or —N═N-L⁴. L¹, L², L³ and L⁴ are as defined in L¹, L², L³ and L⁴ in the formulas (I-2-1) and (I-2-2), so that the detailed description will be omitted here. The amino group, the amide group, the imide group and the silyl group can be each substituted by the group(s) L.

—CₘHₘ₊₁ (q1)

—CₐHₐ₊₁—OC_{b}H_{b+1} (q2)

In the formula (q1), m represents an integer of 1 to 5. In the formula (q2), a and b each represent an integer of 1 to 5.

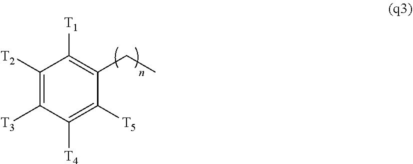

In the formula (q3), n represents an integer of 1 to 5; T₁, T₂, T₃, T₄ and T₅ each independently represent a hydrogen atom or —OCₚHₚ₊₁; and p represents an integer of 1 to 5.

Each of the aliphatic hydrocarbon group having 1 to 12 carbon atoms, the halogen-substituted alkyl group having 1 to 12 carbon atoms, the alicyclic hydrocarbon group having 3 to 14 carbon atoms, the aromatic hydrocarbon group having 6 to 14 carbon atoms, and the heterocyclic group having 3 to 14 carbon atoms, each of which optionally has the group(s) L, preferably has a total of 50 or less carbon atoms, more preferably 40 or less carbon atoms, particularly preferably 30 or less carbon atoms, including substituent(s). When the number of carbon atoms falls within this range, the cyanine compound is more easily synthesized, while absorption intensity per unit weight tends to be high.

Examples of the aliphatic hydrocarbon group having 1 to 12 carbon atoms, the halogen-substituted alkyl group having 1 to 12 carbon atoms, the alicyclic hydrocarbon group having 3 to 14 carbon atoms, the aromatic hydrocarbon group having 6 to 14 carbon atoms, the heterocyclic group having 3 to 14 carbon atoms, and the group(s) L include the same as those listed above, so that the description will be omitted here.

Examples of —S-L² include a thiol group, a methyl sulfide group, an ethyl sulfide group, a propyl sulfide group, a butyl sulfide group, an isobutyl sulfide group, a sec-butyl sulfide group, a tert-butyl sulfide group, a phenyl sulfide group, a 2,6-di-tert-butylphenyl sulfide group, a 2,6-diphenylphenyl sulfide group, and a 4-cumylphenyl sulfide group.

Examples of —SS-L² include a disulfide group, a methyl disulfide group, an ethyl disulfide group, a propyl disulfide group, a butyl disulfide group, an isobutyl disulfide group, a sec-butyl disulfide group, a tert-butyl disulfide group, a phenyl disulfide group, a 2,6-di-tert-butylphenyl disulfide group, a 2,6-diphenylphenyl disulfide group, and a 4-cumylphenyl disulfide group.

Examples of —SO₂-L³ include a sulfoxyl group, a mesyl group, an ethylsulfonyl group, a n-butylsulfonyl group, and a p-toluenesulfonyl group.

Examples of —N═N-L⁴ include a methylazo group, a phenylazo group, a p-methylphenylazo group, and a p-dimethylaminophenylazo group.

The anion in the cyanine compound of the present embodiment can be prepared in accordance with a method described in Examples mentioned later or with reference to the method. The cation in the cyanine compound of the present embodiment can be prepared by a heretofore known method.

The cyanine compound of the present embodiment, particularly, by having the anion mentioned above, easily has a local maximum absorption wavelength of more than 800 nm. This facilitates more selectively absorbing an incident light (particularly, infrared light) of more than 800 nm, while suppressing the absorption of visible light. This is presumably because the anion mentioned above has a structure that easily narrows an energy gap, though the factor is not limited thereto. Furthermore, the cyanine compound of the present embodiment, particularly, by having the anion mentioned above, easily exhibits higher durability performance (e.g., light resistance and heat resistance). This is presumably because the anion mentioned above has a more stabilized molecular orbital and because, particularly, when R¹ to R⁴ are bulky, active oxygen or the like is inhibited from coming close to a methine site which is susceptible spontaneous oxidation so that the deterioration of the anion is suppressed, though the factor is not limited thereto.

(Photoelectric Conversion Element)

The photoelectric conversion element of the present embodiment refers to an element that has a photoelectric conversion unit which generates a charge in response to the quantity of an incident light in the infrared region (hereinafter, also referred to as an infrared photoelectric conversion unit), and outputs the generated charge to the outside of the photoelectric conversion element through a condenser (also referred to as an accumulation unit) for charge accumulation, a transistor circuit (also referred to as a readout unit) for readout, and the like. In this context, the infrared photoelectric conversion unit refers to a unit having an organic infrared photoelectric conversion film disposed between a pair of opposed electrodes, and a light is incident on the photoelectric conversion unit from above the electrodes. The organic infrared photoelectric conversion film is a photosensitive thin film containing a material that absorbs at least a portion of incident lights in the infrared region (hereinafter, referred to as an “organic infrared absorptive material”), and generates holes and electrons as a result of light incidence.

(Organic Infrared Absorptive Material)

The organic infrared absorptive material according to the present embodiment contains the cyanine compound mentioned above.

In the organic infrared absorptive material according to the present embodiment, the local maximum absorption wavelength and the maximum absorption wavelength of optical absorption spectra in the infrared region are preferably 800 nm or more and 2500 nm or less. Specifically, an organic photoelectric conversion film in the form of a thin film prepared from the organic infrared absorptive material has light absorption peaks that exhibit local maximum and maximum values in the wavelength range of 800 nm to 2500 nm. In this range, the absorption ratio of an infrared light absorption peak is preferably 50% or more.

The organic infrared absorptive material according to the present embodiment preferably has no or minimum absorption in a wavelength region other than 800 nm to 2500 nm. However, the organic infrared absorptive material according to the present embodiment exhibits a local maximum absorption wavelength and a maximum absorption wavelength of optical absorption spectra in the infrared region of 800 nm or more and 2500 nm or less, and can achieve the absorption of the wavelength in a solid-phase state when used in the photoelectric conversion element. In general, a photoelectric conversion material for use in photoelectric conversion elements can improve sensitivity as its molar extinction coefficient is higher. Therefore, a higher molar extinction coefficient is preferred.

The organic infrared absorptive material according to the present embodiment may be composed of only the cyanine compound or may contain an additional infrared absorptive substance known in the art. Examples of such a compound include cyanine compounds other than the compound described above, squarylium compounds, croconium compounds, immonium compounds, dithiolene compounds, bisdithiolene compounds, porphyrin compounds, phthalocyanine compounds, naphthalocyanine compounds, BODIPY compounds, and quaterrylene diimide.

(Organic Infrared Photoelectric Conversion Film)

The organic infrared photoelectric conversion film for use in the photoelectric conversion element of the present embodiment can be obtained, for example, by preparing the organic infrared absorptive material into a thin film, Examples of the method for forming the organic infrared photoelectric conversion film according to the present embodiment include general dry film formation methods and wet film formation methods. Specific examples of such a formation method include: resistance heating vapor deposition, electron beam vapor deposition, sputtering, and molecular lamination methods which are vacuum processes; casting which is a solution process; coating methods such as spin coating, dip coating, blade coating, wire bar coating, and spray coating; printing methods such as inkjet printing, screen printing, offset printing, and relief printing; and soft lithography approaches such as microcontact printing methods. A combined method of a plurality of these approaches may be adopted for the film formation of each layer.

For example, in the dry film formation, the cyanine compound of the present embodiment, and optionally, a compound appropriate for the application of the photoelectric conversion element are mixed to prepare a composition, which can then be vapor-deposited onto an electrode or an organic thin film layer mentioned later in vacuum to obtain an organic infrared photoelectric conversion film. In the wet film formation method, the cyanine compound of the present embodiment, and optionally, a compound appropriate for the application of the photoelectric conversion element are mixed together with a solvent to prepare a liquid composition, with which an electrode or an organic thin film can then be coated or printed, followed by drying to obtain an organic infrared photoelectric conversion film.

The thickness of the organic infrared photoelectric conversion film prepared so as to contain the cyanine compound depends on the resistance value and/or charge mobility of each substance and thus cannot be limited. The thickness is usually 0.5 nm or more and 5000 nm or less, preferably 1 nm or more and 1000 nm or less, more preferably 5 nm or more and 500 nm or less.

The organic infrared photoelectric conversion film according to the present embodiment may contain an organic material other than the cyanine compound and preferably contains a p-type and/or n-type organic semiconductor because incident light energy can be more efficiently converted to electric signals. Among others, an organic p-type semiconductor that easily donates electrons (which has a small ionization potential) to the organic infrared absorptive material, or an organic n-type semiconductor that easily accepts electrons therefrom (which has large electron affinity) is preferred because incident light energy can be still more efficiently converted to electric signals. More specifically, the ionization potential (HOMO level) is preferably −5.5 eV or more in terms of a thin-film solid. The electron affinity (LUMO level) is preferably −3.0 eV or less in terms of a thin-film solid. In this context, the ionization potential (HOMO level) refers to a value measured by photoemission yield spectroscopy in air. The electron affinity (LUMO level) refers to a value determined by calculating an energy band gap value from the absorption end of the longest wavelength of near-infrared spectra, and subtracting the value from the HOMO level.

In the case of using an organic semiconductor, a form in which the cyanine compound of the present embodiment and the organic semiconductor are used as a mixture, and a multilayer form in which a layer prepared from only the cyanine compound of the present embodiment (hereinafter, referred to as a “cyanine compound layer”) and a layer prepared from only the organic semiconductor (hereinafter, referred to as an “organic semiconductor layer”) are used may both be adopted.

In the case of using an organic semiconductor layer, the layer may be one layer or may be two or more layers. The organic semiconductor layer may be an organic p-type semiconductor film, may be an organic n-type semiconductor film, or may be a mixed film thereof (bulk-hetero structure). Particularly, the organic semiconductor layer preferably has a layer having a bulk-hetero junction structure. In such a case, the organic infrared photoelectric conversion film is allowed to have a bulk-hetero junction structure. This can compensate for the disadvantage, i.e., a short carrier diffusion length, of the organic infrared photoelectric conversion film and improve photoelectric conversion efficiency.

In the case of using the cyanine compound layer and the organic semiconductor layer in combination, the thickness of a laminate of these layers depends on the resistance value and/or charge mobility of each substance and thus cannot be limited. The thickness is usually 0.5 nm or more and 5000 nm or less, preferably 1 nm or more and 1000 nm or less, more preferably in the range of 5 to 500 nm. In this case, the organic semiconductor layer is preferably on the order of two or more layers and 10 or less layers.

Hereinafter, the organic semiconductor will be described in detail.

(Organic p-Type Semiconductor)

The organic p-type semiconductor (compound) is a donor organic semiconductor (hereinafter, also referred to as a “donor organic compound”) and refers to an organic compound that is typified mainly by a hole-transporting organic compound and has a property of easily donating electrons. Further specifically, this compound refers to an organic compound having a smaller ionization potential when two organic materials are used in contact. Thus, any organic compound may be used as the donor organic compound as long as the organic compound has electron-donating properties.

Examples of such a donor organic compound include triarylamine compounds, benzidine compounds, pyrazoline compounds, styrylamine compounds, hydrazone compounds, triphenylmethane compounds, carbazole compounds, polysilane compounds, thiophene compounds, phthalocyanine compounds, cyanine compounds, merocyanine compounds, oxonol compounds, polyamine compounds, indole compounds, pyrrole compounds, pyrazole compounds, polyarylene compounds, condensed aromatic carbocyclic compounds (naphthalene derivatives, anthracene derivatives, phenanthrene derivatives, tetracene derivatives, pyrene derivatives, perylene derivatives, and fluoranthene derivatives), and metal complexes having nitrogen-containing heterocyclic compounds as ligands. The donor organic compound is not limited thereto, and as described above, an organic compound having a smaller ionization potential than that of an organic compound used as an acceptor organic compound may be used as the donor organic semiconductor.

(Organic n-Type Semiconductor)

The organic n-type semiconductor (compound) is an acceptor organic semiconductor (hereinafter, also referred to as an “acceptor organic compound”) and refers to an organic compound that is typified mainly by an electron-transporting organic compound and has a property of easily accepting electrons. Further specifically, this compound refers to an organic compound having larger electron affinity when two organic compounds are used in contact. Thus, any organic compound may be used as the acceptor organic compound as long as the organic compound has electron-accepting properties.

Examples of such an acceptor organic compound include condensed aromatic carbocyclic compounds (naphthalene derivatives, anthracene derivatives, phenanthrene derivatives, tetracene derivatives, pyrene derivatives, perylene derivatives, fluoranthene derivatives, and fullerene derivatives), 5- to 7-membered heterocyclic compounds containing a nitrogen atom, an oxygen atom, or a sulfur atom (e.g., pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, thiazole, oxazole, indazole, benzimidazole, benzotriazole, benzoxazole, benzothiazole, carbazole, purine, triazolopyridazine, triazolopyrimidine, tetrazaindene, oxadiazole, imidazopyridine, pyralidine, pyrrolopyridine, thiadiazolopyridine, dibenzazepine, and tribenzazepine), polyarylene compounds, fluorene compounds, cyclopentadiene compounds, silyl compounds, and metal complexes having nitrogen-containing heterocyclic compounds as ligands. The acceptor organic compound is not limited thereto, and as described above, an organic compound having larger electron affinity than that of an organic compound used as a donor organic compound may be used as the acceptor organic semiconductor.

(Infrared Photoelectric Conversion Unit)

The infrared photoelectric conversion unit according to the present embodiment has a pair of electrodes and the organic infrared photoelectric conversion film disposed between the pair of electrodes. This infrared photoelectric conversion unit may employ an organic thin film layer in addition to the pair of electrodes and the organic infrared photoelectric conversion film. This infrared photoelectric conversion unit may have, for example, an electron transport layer, a hole transport layer, an electron blocking layer, a hole blocking layer, an electron injection layer, a hole injection layer, a crystallization prevention layer, and/or an interlayer contact improvement layer, as a layer other than the organic infrared photoelectric conversion film. Particularly, the infrared photoelectric conversion unit having one or more selected from the group consisting of an electron transport layer, a hole transport layer, an electron blocking layer and a hole blocking layer is preferred because the resulting element more efficiently converts even weak light energy to electric signals.

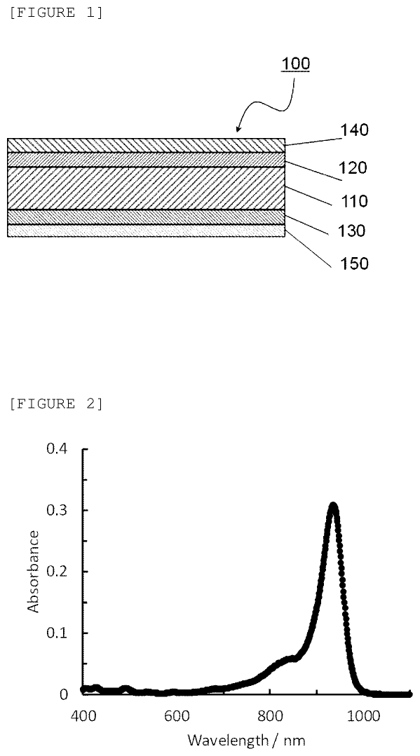

FIG. 1 is a cross-sectional schematic view partially showing one example of the infrared photoelectric conversion unit according to the present embodiment. An infrared photoelectric conversion unit 100 shown in FIG. 1 has an organic infrared photoelectric conversion film 110 containing an organic infrared absorptive material, a hole transport layer 120 and an electron transport layer 130 laminated so as to flank the organic infrared photoelectric conversion film 110, and electrodes 140 and 150 laminated so as to further flank the resultant. This infrared photoelectric conversion unit 100 can selectively cause photoelectric conversion of an infrared wavelength of 800 nm or more among incident lights including visible light and infrared light, principally due to the organic infrared photoelectric conversion film containing an organic infrared absorptive material. Hereinafter, each member possessed by the infrared photoelectric conversion unit 100 will be described in detail.

(Electrode)

When the organic infrared photoelectric conversion film contained in the infrared photoelectric conversion unit has hole-transporting properties or when an organic thin film layer other than the organic infrared photoelectric conversion film is a hole transport layer having hole-transporting properties, the electrode plays a role in extracting holes from the organic infrared photoelectric conversion film or the additional organic thin film layer and collecting the holes. When the organic infrared photoelectric conversion film contained in the infrared photoelectric conversion unit has electron-transporting properties or when an organic thin film layer other than the organic infrared photoelectric conversion film is an electron transport layer having electron-transporting properties, the electrode plays a role in extracting electrons from the organic infrared photoelectric conversion film or the additional organic thin film layer and discharging the electrons.

The material that may be used in the electrode is not particularly limited as long as it has conductivity to some extent. The material is preferably selected in consideration of close contact with the adjacent organic infrared photoelectric conversion film or additional organic thin film layer, electron affinity, an ionization potential and stability, etc. Examples of the material that may be used in the electrode include: conductive metal oxides such as tin oxide (NESA), indium oxide, indium tin oxide (ITO) and indium zinc oxide (IZO); metals such as gold, silver, platinum, chromium, aluminum, iron, cobalt, nickel and tungsten: inorganic conductive substances such as copper iodide and copper sulfide; conductive polymers such as polythiophene, polypyrrole and polyaniline; and carbon. These materials may each be used singly, may be used as a mixture of two or more thereof, or may be used as a two-layer or more laminate of two or more thereof. The thickness of the electrode can be arbitrarily selected in consideration of conductivity. The thickness may be 5 nm or more and 500 nm or less and is preferably 10 nm or more and 300 nm or less.

The conductivity of the material for use in the electrode is not particularly limited as long as it does not hinder the light reception of the photoelectric conversion element more than necessary. The conductivity is preferably as high as possible from the viewpoint of the signal intensity and power consumption of the photoelectric conversion element. For example, as for a transparent electrode, an ITO film having conductivity equal to or less than a sheet resistance value of 300 ohms per square sufficiently functions as the electrode. However, a commercially available product of a substrate having an ITO film having conductivity on the order of several ohms per square (e.g., 5 to 9 ohms per square) is also obtainable, and such a substrate having high conductivity is desirable.

In the case of using an ITO film, the thickness of the electrode can be arbitrarily selected in consideration of conductivity and is usually 5 nm or more and 3000 nm or less, preferably 10 nm or more and 300 nm or less. Examples of the method for forming the film such as ITO include vapor deposition methods, electron beam methods, sputtering methods, chemical reaction methods and coating methods heretofore known in the art. The ITO film disposed on the substrate may be subjected, if necessary, to UV-ozone treatment or plasma treatment.

In the case of laminating a plurality of organic infrared photoelectric conversion films differing in wavelength to be detected, the film of an electrode (which is the film of an electrode other than the pair of electrodes described above) for use between the respective organic infrared photoelectric conversion films needs to be transmissive to a light having a wavelength other than the lights to be detected by the respective organic infrared photoelectric conversion films. From such a viewpoint, a material transmissive to 90% more of an incident light is preferably used in the film of the electrode, and a material transmissive to 95% or more of a light is more preferably used.

In the case of further establishing a photoelectric conversion unit that senses a light in the visible light region beneath the infrared photoelectric conversion unit according to the present embodiment, the electrode for use in the infrared photoelectric conversion unit preferably has a transmittance of 90% or more, more preferably 95% or more, to visible light and infrared light.

The material for the electrode that satisfies such conditions is preferably transparent conducting oxide (TCO) having a high transmittance to visible light and infrared light and a small resistance value. Although a thin film of a metal such as Au can be used as an electrode, its resistance value is extremely increased if the transmittance is adjusted to 90% or more. Thus, TCO is preferred for the electrode. The TCO is particularly preferably ITO, IZO, AZO, FTO, SnO₂, TiO₂ or ZnO₂.

The method for forming the electrode is not particularly limited and can be appropriately selected in consideration of aptitude for the electrode material. In the case of using a transparent electrode, specific examples of the formation method therefor include wet schemes such as printing schemes and coating schemes, physical schemes such as vacuum vapor deposition methods, sputtering methods and ion plating methods, and chemical schemes such as CVD and plasma CVD. When the electrode material is transparent conducting metal oxide such as ITO, examples of the formation method therefor include electron beam methods, sputtering methods, resistance heating vapor deposition methods, chemical reaction methods (sol-gel methods, etc.), and methods of coating with a dispersion of the metal oxide. The film of transparent conducting metal oxide such as ITO may be further subjected to UV-ozone treatment and plasma treatment.

Next, the organic thin film layer other than the organic infrared photoelectric conversion film will be described.

The electron transport layer plays a role in transporting electrons generated in the organic infrared photoelectric conversion film to the electrode, and a role in blocking hole migration to the organic infrared photoelectric conversion film from the electrode to which electrons are transported.

The hole transport layer plays a role in transporting generated holes from the organic infrared photoelectric conversion film to the electrode, and a role in blocking electron migration to the organic infrared photoelectric conversion film from the electrode to which holes are transported.

The electron blocking layer plays a role in blocking electron migration from the electrode to the organic infrared photoelectric conversion film, preventing electron recombination in the organic infrared photoelectric conversion film, reducing dark current, reducing noise, and expanding a dynamic range.

The hole blocking layer plays a role in blocking hole migration from the electrode to the organic infrared photoelectric conversion film, preventing hole recombination in the organic infrared photoelectric conversion film, reducing dark current, reducing noise, and expanding a dynamic range.

(Hole Transport Layer)

The material for the hole transport layer is not particularly limited as long as it is known as a hole transport layer for photoelectric conversion elements such as solid image sensors. Examples thereof include polyaniline and doped materials thereof, and cyanogen compounds described in International Publication No. WO 2006/019270.

More specific examples of the material constituting the hole transport layer include selenium, iodides such as copper iodide (CuI), cobalt complexes such as layered cobalt oxide, CuSCN, molybdenum oxide (MoO₃, etc.), nickel oxide (NiO, etc.), 4CuBr·3S(C₄H₉) and organic hole transport materials. Among them, examples of the iodide include copper iodide (CuI). Examples of the layered cobalt oxide include AxCoO₂ (wherein A represents Li, Na, K, Ca, Sr or Ba, and 0≤X≤1).

Examples of the organic hole transport material include polythiophene derivatives such as poly-3-hexylthiophene (P3HT) and poly(3,4-ethylenedioxythiophene) (PEDOT; e.g., trade name “BaytronP” manufactured by H.C. Starck-V Tech Ltd.), fluorene derivatives such as 2,2′, 7,7′-tetrakis-(N,N-di-p-methoxyphenylamine)-9,9′-spirobifluorene (spiro-MeO-TAD), carbazole derivatives such as polyvinylcarbazole, triphenylamine derivatives, diphenylamine derivatives, polysilane derivatives, and polyaniline derivatives. Further examples of the material for the hole transport layer include compound semiconductors having monovalent copper, such as CuInSe₂ and copper sulfide (CuS), gallium phosphide (GaP), nickel oxide (NiO), cobalt oxide (CoO), iron oxide (FeO), bismuth oxide (Bi₂O₃), molybdenum oxide (MoO₂), and chromium oxide (Cr₂O₃).

The hole transport layer having a shallower LUMO level than that of the organic infrared photoelectric conversion film is preferred because an electron blocking function having a rectifying effect of suppressing the migration of electrons generated in the organic infrared photoelectric conversion film to the electrode side is imparted thereto. Such a hole transport layer is also called an electron blocking layer.

Examples of the low-molecular organic compound as the material constituting the electron blocking layer include aromatic diamine compounds such as N,N′-bis(3-methylphenyl)-(1,1′-biphenyl)-4,4′-diamine (TPD) and 4,4′-bis[N-(naphthyl)-N-phenyl-amino]biphenyl (α-NPD), oxazole, oxadiazole, triazole, imidazole, imidazolone, stilbene derivatives, pyrazoline derivatives, tetrahydroimidazole, polyarylalkane, butadiene, 4,4′,4″-tris(N-(3-methylphenyl)N-phenylamino)triphenylamine (m-MTDATA), porphyrin compounds such as porphyrin, copper tetraphenylporphyrin, phthalocyanine, copper phthalocyanine and titanium phthalocyanine oxide, triazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, and silazanes derivatives. Examples of the high-molecular organic compound include polymers such as phenylene vinylene, fluorene, carbazole, indole, pyrene, pyrrole, picoline, thiophene, acetylene and diacetylene, and derivatives thereof. A compound having sufficient hole-transporting properties, albeit being not an electron-donating compound, may be used as the material constituting the electron blocking layer. Examples of the inorganic compound as the material constituting the electron blocking layer include metal oxides such as calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide and iridium oxide, selenium, tellurium and antimony sulfide. These materials may each be used singly or in combination of two or more thereof.

The thickness of the hole transport layer is preferably 10 nm or more and 300 nm or less, more preferably 30 nm or more and 250 nm or less, further preferably 50 nm or more and 200 nm or less, from the viewpoint of suppressing dark current, and preventing reduction in photoelectric conversion efficiency.

The method for forming the hole transport layer may be a heretofore known method and may be any of dry film formation methods such as vacuum vapor deposition methods, and wet film formation methods such as solution coating methods. A wet film formation method is preferred from the viewpoint of being able to level a coated surface. Examples of the dry film formation method include vapor deposition methods such as vacuum vapor deposition methods, and sputter methods. The vapor deposition may be any of physical vapor deposition (PVD) and chemical vapor deposition (CVD) and is preferably physical vapor deposition such as vacuum vapor deposition. Examples of the wet film formation method include inkjet methods, spray methods, nozzle print methods, spin coating methods, dip coating methods, casting methods, die coating methods, roll coating methods, bar coating methods and gravure coating methods.

(Electron Transport Layer)

The material constituting the electron transport layer is not particularly limited as long as it is known as an electron transport layer for photoelectric conversion elements such as solid image sensors. Examples thereof include organic compounds such as perfluoro forms (perfluoropentacene, perfluorophthalocyanine, etc.) of octaazaporphyrin and p-type semiconductors, fullerene, fullerene derivatives (e.g., [6,6]-phenyl-C61-butyric acid methyl ester; PCBM), perylene, indenoindene and indenoindene derivatives, and inorganic oxides such as titanium oxide (TiO₂, etc.), nickel oxide (NiO), tin oxide (SnO₂), tungsten oxide (WO₂, WO₃, W₂O₃, etc.), zinc oxide (ZnO), niobium oxide (Nb₂O₅, etc.), tantalum oxide (Ta₂O₅, etc.), yttrium oxide (Y₂O₃, etc.), and strontium titanate (SrTiO₃, etc.). The electron transport layer may be a porous layer or may be a dense layer. In the case of laminating them, the porous electron transport layer and the dense electron transport layer are preferably laminated and disposed in this order from the organic infrared photoelectric conversion film side.

The electron transport layer having a deeper HOMO level than that of the organic infrared photoelectric conversion film is preferred because a hole blocking function having a rectifying effect of suppressing the migration of holes generated in the organic infrared photoelectric conversion film to the opposite electrode side is imparted thereto. Such an electron transport layer is also called a hole blocking layer.

Examples of the material constituting the hole blocking layer include oxadiazole derivatives such as 1,3-bis(4-tert-butylphenyl-1,3,4-oxadiazolyl)phenylene (OXD-7), anthraquinone dimethane derivatives, diphenylquinone derivatives, bathocuproine, bathophenanthroline, and their derivatives, triazole compounds, tris(8-hydroxyquinolinato)aluminum complexes, bis(4-methyl-8-quinolinato)aluminum complexes, silole compounds, porphyrin compounds, styryl compounds such as DCM (4-dicyanomethylene-2-methyl-6-(4-(dimethylaminostyryl))-4H-pyran), n-type semiconductor materials such as naphthalenetetracarboxylic anhydrides, naphthalenetetracarboxylic acid diimide, perylenetetracarboxylic anhydrides, and perylenetetracarboxylic acid diimide, n-type inorganic oxides such as titanium oxide, zinc oxide and gallium oxide, and alkali metal fluorides such as lithium fluoride, sodium fluoride and cesium fluoride. Further, an alkali metal compound doped with an organic semiconductor molecule is also preferred because of having a function of improving electric junction to the opposite electrode. These materials may each be used singly or in combination of two or more thereof.

The thickness of the electron transport layer is preferably 10 nm or more and 300 nm or less, more preferably 30 nm or more and 250 nm or less, further preferably 50 nm or more and 200 nm or less, from the viewpoint of suppressing dark current, and preventing reduction in photoelectric conversion efficiency.

The method for forming the electron transport layer may be a heretofore known method and may be any of dry film formation methods such as vacuum vapor deposition methods, and wet film formation methods such as solution coating methods. A wet film formation method is preferred from the viewpoint of being able to level a coated surface. Examples of the dry film formation method include vapor deposition methods such as vacuum vapor deposition methods, and sputter methods. The vapor deposition may be any of physical vapor deposition (PVD) and chemical vapor deposition (CVD) and is preferably physical vapor deposition such as vacuum vapor deposition. Examples of the wet film formation method include inkjet methods, spray methods, nozzle print methods, spin coating methods, dip coating methods, casting methods, die coating methods, roll coating methods, bar coating methods and gravure coating methods.

(Interlayer Contact Improvement Layer)

The interlayer contact improvement layer has a function of reducing damage on a lower film nearest to an upper electrode, for example, the organic infrared photoelectric conversion film, at the time of film formation of the upper electrode. Particularly, high-energy particles present in an apparatus for use in the film formation of the upper electrode to be formed, for example, sputter particles, secondary electrons, Ar particles, or oxygen anions in a sputtering method, may deteriorate the lower film nearest thereto through collision, resulting in performance deterioration such as increased leak current or reduced sensitivity. One of the methods for preventing this preferably involves establishing an interlayer contact improvement layer on an upper layer of the nearest lower film. An organic material such as copper phthalocyanine, PTCDA, an acetyl acetonate complex, or BCP, an organic metal compound, or an inorganic material such as MgAg or MgO is preferably used as the material for the interlayer contact improvement layer. The thickness of the interlayer contact improvement layer differs in proper range depending on the configuration of a photoelectric conversion film, the film thickness of an electrode, etc. It is particularly preferred to select a material having no absorption in the visible region, or the thickness is preferably 2 nm or more and 50 nm from the viewpoint of using a very small thickness.

The photoelectric conversion element of the present embodiment has an infrared photoelectric conversion unit that generates a charge in response to the quantity of an incident light in the infrared region. The generated charge is read out as signals depending on the quantity of the charge by a semiconductor. Hence, the photoelectric conversion element is connected with a condenser for accumulation of the generated charge (hereinafter, also referred to as an “accumulation unit”) and a transistor circuit for readout (hereinafter, also referred to as a “readout unit”) via a connection unit made of a conductive material. Also, the photoelectric conversion element optionally contains a substrate for strength retention, a microlens for light collection, or the like.

(Accumulation Unit, Readout Unit, and Connection Unit)

The readout unit is disposed in order to read out signals depending on a charge generated in the organic infrared photoelectric conversion film. The readout unit is constituted by, for example, CCD, a CMOS circuit, or a TFT circuit, and preferably shielded from lights by a light shielding layer disposed in an insulating layer. The readout circuit is electrically connected with its corresponding electrode via a connection unit. In order to secure a charge in a quantity necessary for readout, an accumulation unit constituted by a condenser or the like may intervene between the electrode and the connection unit. The connection unit is embedded in the insulating layer and is a plug or the like for electrically connecting the electrode (e.g., a transparent electrode or an opposite electrode) to the readout unit. When the member thus configured is a solid image sensor, upon light incidence, the light is incident on the organic infrared photoelectric conversion film where a charge is then generated. Electrons in the generated charge are collected (and accumulated) in one electrode, and voltage signals depending on the quantity thereof is output to the outside of the solid image sensor by the readout unit.

(Visible Photoelectric Conversion Unit)

The photoelectric conversion element of the present embodiment preferably has a visible photoelectric conversion unit having absorption spectra in the visible light region, from the viewpoint of improving photoelectric conversion sensitivity, and from the viewpoint of improving an image processing rate when infrared imaging or positional information using infrared light is used in combination with visible imaging. This visible photoelectric conversion unit can be disposed beneath the infrared photoelectric conversion unit in order to simultaneously sense visible light and infrared light by the photoelectric conversion of a transmitted light in the visible light region when the infrared photoelectric conversion unit is transmissive to a light in the visible region, for example, when the photoelectric conversion element of the present embodiment has a transparent electrode.

The visible photoelectric conversion unit may sense a light in the visible light region by using a heretofore known silicon photodiode or a device having an organic photoelectric conversion material sensitive to visible light (e.g., those described in Japanese Patent Application Laid-Open No. 2013-258168). For color imaging, a color filter or the like may be disposed above the visible photoelectric conversion unit, or the visible photoelectric conversion unit may be laminated with an organic photoelectric conversion layer differing in visible light wavelength sensitivity therefrom.

The photoelectric conversion element of the present embodiment, particularly, by having the organic infrared photoelectric conversion film containing the cyanine compound having the anion mentioned above, is facilitated to more selectively absorb an incident light (particularly, infrared light) of more than 800 nm, and is consequently excellent in photoelectric conversion efficiency. This is presumably because the anion mentioned above has a structure that easily narrows an energy gap, though the factor is not limited thereto. Furthermore, the photoelectric conversion element of the present embodiment, particularly, by having the organic infrared photoelectric conversion film containing the cyanine compound having the anion mentioned above, easily exhibits higher durability performance (e.g., light resistance and heat resistance). This is presumably because the anion mentioned above has a more stabilized molecular orbital, though the factor is not limited thereto.

(Solid Image Sensor)

The solid image sensor of the present embodiment has a large number of photoelectric conversion elements of the present embodiment disposed in an array pattern. Specifically, a large number of photoelectric conversion elements disposed in an array pattern constitute a solid image sensor that exhibits the quantity of an incident light as well as positional information on incidence.

In the solid image sensor, a plurality of photoelectric conversion units may be laminated as long as an infrared photoelectric conversion unit disposed nearer a light source does not block (or is transmissive to) the absorption wavelength of another photoelectric conversion unit (visible photoelectric conversion unit, etc.) disposed at the back thereof viewed from the light source side.

In the solid image sensor, the infrared photoelectric conversion unit or the visible photoelectric conversion unit may be partially constituted as a thin film in the same plane having no structural separation between the adjacent photoelectric conversion elements, from the viewpoint of easy molding.

The solid image sensor of the present embodiment may further include a substrate. The substrate is used for producing the solid image sensor by laminating each layer thereon, or used for enhancing the mechanical strength of the solid image sensor. Examples of the type of the substrate include, but are not particularly limited to, semiconductor substrates, glass substrates and plastic substrates.

### EXAMPLES

Hereinafter, the present invention will be described in more detail with reference to Examples. However, the present invention is not limited by these Examples.

### Example 1

First, compound 1a was synthesized in accordance with the following scheme.

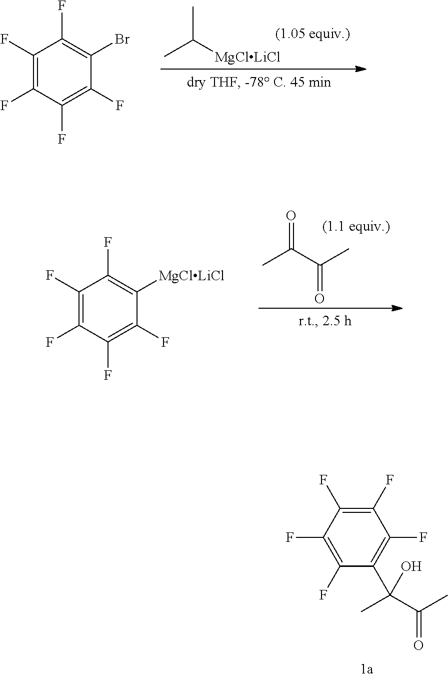

To a solution of an isopropyl magnesium chloride-lithium chloride complex in dry tetrahydrofuran (approximately 14%), bromopentafluorobenzene was added such that the molar ratio between the complex and the bromopentafluorobenzene was 1.05:1.00. The mixture was reacted by stirring at −78° C. for 45 minutes. To the obtained product, 2,3-butanedione was added such that the molar ratio between the product and the 2,3-butanedione was 1.0:1.1. The mixture was reacted by stirring at room temperature for 2.5 hours to obtain compound 1a. The obtained compound 1a was purified, and its yield was measured and was consequently 53%.

Next, tricyanofuran fluoride 2a was synthesized in accordance with the following scheme. The tricyanofuran fluoride 2a was synthesized with reference to Chem, Mater. 2002, 14, p. 2393-2400.

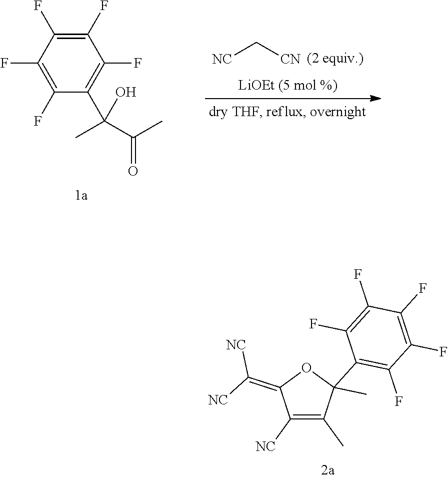 [SMILES: CC(=O)C(C)(O)c1c(F)c(F)c(F)c(F)c1F ; N#CCC#N ; CC1=C(C#N)C(=C(C#N)C#N)OC1(C)c1c(F)c(F)c(F)c(F)c1F]

A 1 M ethanol solution supplemented with 5 mol % of lithium ethoxide, and malononitrile were added to dry tetrahydrofuran, and further, the compound 1a was added thereto such that the molar ratio between the malononitrile and the compound 1a was 2:1. The mixture was reacted by reflux overnight to obtain tricyanofuran fluoride 2a. The obtained tricyanofuran fluoride 2a was purified, and its yield was measured and was consequently 10%.

Also, dialdehyde 3 known in the art was synthesized in accordance with the following scheme.

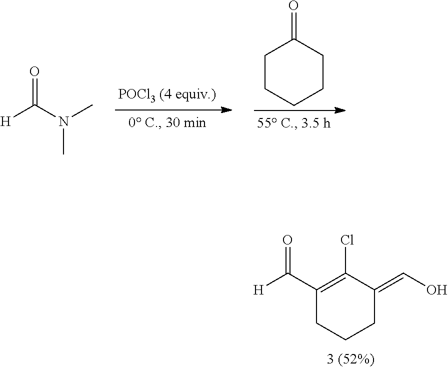

Excess dimethylformamide and 4 equivalents of phosphoryl chloride based on cyclohexanone were mixed and reacted at 0° C. for 30 minutes. To the obtained product, cyclohexanone was added, and the mixture was further reacted by stirring at 55° C. for 3.5 hours to obtain dialdehyde 3. The obtained dialdehyde 3 was purified, and its yield was measured and was consequently 52%.

Further, cyanine compound 4a having a perfluorophenyl group was synthesized in accordance with the following scheme.

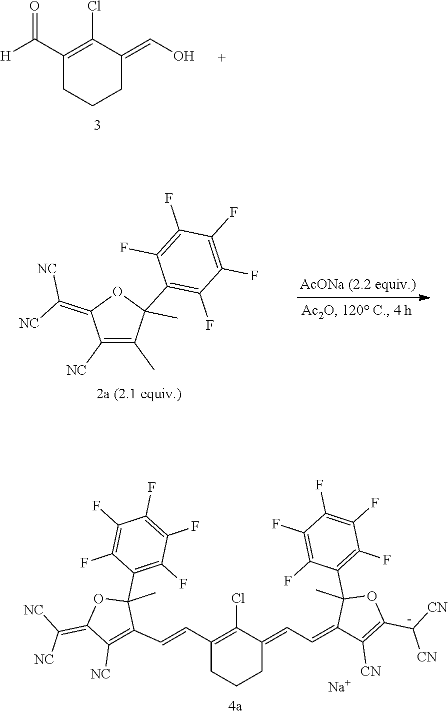 [SMILES: O=CC1=C(Cl)/C(=C/O)CCC1 ; "+" ; CC1=C(C#N)C(=C(C#N)C#N)OC1(C)c1c(F)c(F)c(F)c(F)c1F ; CC1(c2c(F)c(F)c(F)c(F)c2F)OC(=C(C#N)C#N)C(C#N)=C1/C=C/C1=C(Cl)C(=C/C=C2/C(C#N)=C([C-](C#N)C#N)OC2(C)c2c(F)c(F)c(F)c(F)c2F)/CCC1 ; [Na+]]

The dialdehyde 3 and the tricyanofuran fluoride 2a were added into acetic anhydride supplemented with sodium acetate such that the molar ratio among the dialdehyde 3, the tricyanofuran fluoride 2a, and the sodium acetate was 1.0:2.1:2.2. The mixture was reacted by stirring at 120° C. for 4 hours to synthesize cyanine compound 4a. The obtained cyanine compound 4a was purified, and its yield was measured and was consequently 40%. Results of NMR measurement (measurement apparatus product name: JTM-ECS400, manufactured by JEOL Ltd.; the same holds true for the description below) thereof are shown below.

¹H NMR (Acetone-d₆) δ 1.70-1.76 (m, 2H, —CH₂CH₂CH₂—), 2.16-2.19 (m, 6H, —CH₃ X 2), 2.41-2.56 (m, 4H, —CH₂CH₂CH₂—), 6.14 (d, J=14.2 Hz, 2H, vinyl H), 7.54 (d, J=14.2 Hz, 1H, vinyl H), 7.62 (d, J=14.2 Hz, 1H, vinyl H), ¹⁶F NMR (Acetone-d₆) δ −162.8 (q, J=17.3 Hz, 2F), −153.1 (dt, J=53.5, 21.0 Hz, 1F), −139.3 (d, J=20.2 Hz. 2F).

### Example 2

Cyanine compound 5a having a perfluorophenyl group was synthesized in accordance with the following scheme.

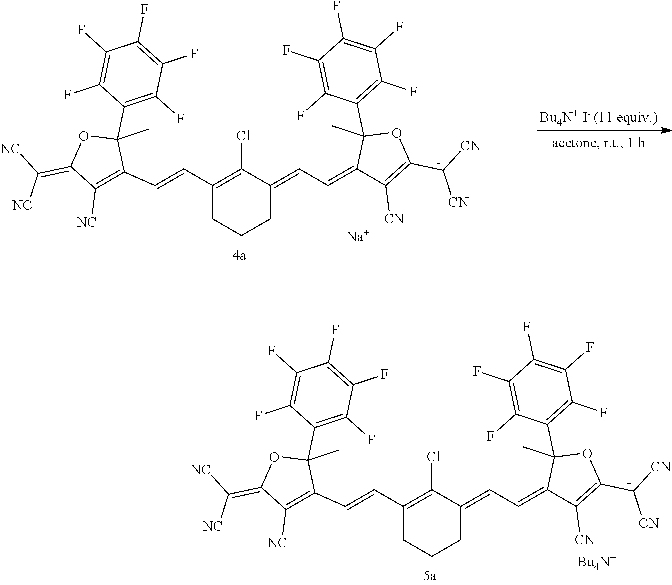

The cyanine compound 4a obtained in Example 1 was added into an acetone solution supplemented with tetrabutylammonium iodide such that the molar ratio between the cyanine compound 4a and the tetrabutylammonium iodide was 1.0:1.1. The mixture was reacted by stirring at room temperature for 1 hour to synthesize cyanine compound 5a. The obtained cyanine compound 5a was purified, and its yield was measured and was consequently 69%. Results of NMR measurement thereof are shown below.

¹H NMR (Acetone-d₆) δ 0.95 (t, J=7.3 Hz, 12H, —CH₂CH₂CH₂CH₃ X 4), 1.36-1.46 (m, 8H, —CH₂CH₂CH₂CH₃ X 4), 1.67-1.73 (m, 2H, —CH₂CH₂CH₂—), 1.76-1.84 (m, 8H, —CH₂CH₂CH₂CH₃ X 4), 2.13-2.17 (m, 6H, —CH₃ X 2), 2.42-2.54 (m, 4H, —CH₂CH₂CH₂—), 3.39-3.44 (m, 8H, —CH₂CH₂CH₂CH₃ X 4), 6.11 (d, J=14.7 Hz, 2H, vinyl H), 7.53 (d, J=14.7 Hz, 1H, vinyl H), 7.66 (d, J=14.7 Hz, 1H, vinyl H), ¹⁹F NMR (Acetone-d₆) δ −162.8 (q, J=16.9 Hz, 2F), −153.1 (dt, J=50.0, 20.2 Hz, 1F), −139.3 (d, J=18.8 Hz, 2F).

### Reference Example 1

Compound 1b known in the art was synthesized in accordance with the following scheme. The compound 1b was synthesized with reference to Angew. Chem, Int. Ed., 2017, 56, p. 2478-2481.

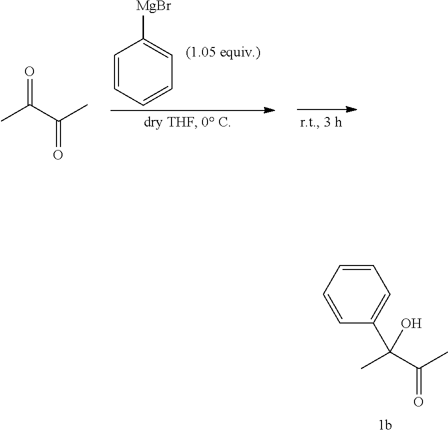

To a solution of phenyl magnesium bromide in dry tetrahydrofuran, 2,3-butanedione was added at 0° C. such that the molar ratio between the phenyl magnesium bromide and the 2,3-butanedione was 1.05:1.00. Then, the mixture was reacted by stirring at room temperature for 3 hours to obtain compound 1b. The crude yield of the obtained compound 1b was measured and was consequently 87%.

Next, tricyanofuran 2b was synthesized in accordance with the following scheme.

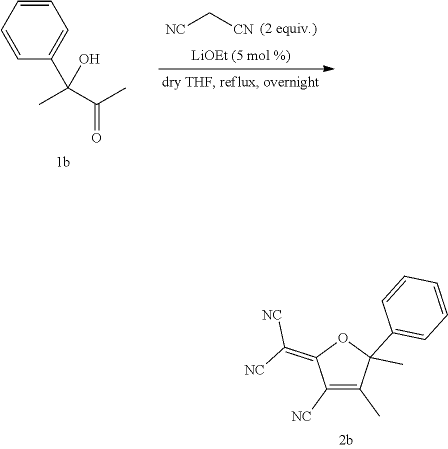 [SMILES: CC(=O)C(C)(O)c1ccccc1 ; N#CCC#N ; CC1=C(C#N)C(=C(C#N)C#N)OC1(C)c1ccccc1]

A 1 M ethanol solution supplemented with 5 mol % of lithium ethoxide, and malononitrile were added to dry tetrahydrofuran, and further, the compound 1b obtained as described above was directly added thereto without being purified such that the molar ratio between the malononitrile and the compound 1b was 2:1. The mixture was reacted by reflux overnight to obtain tricyanofuran 2b. The obtained tricyanofuran 2b was purified, and its yield was measured and was consequently 25%.

Further, cyanine compound 4b was synthesized in accordance with the following scheme.

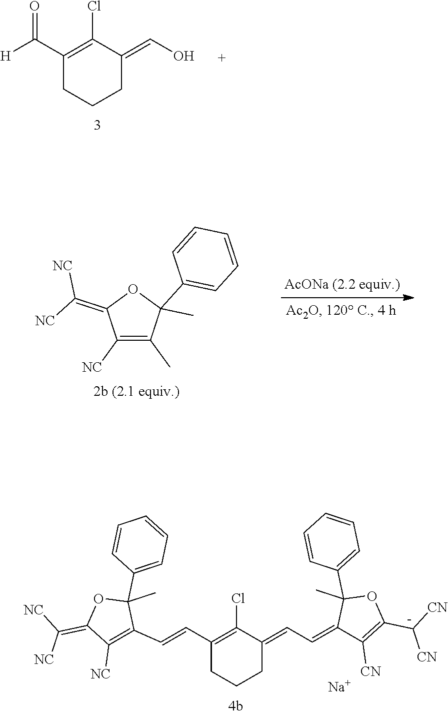 [SMILES: O=CC1=C(Cl)/C(=C/O)CCC1 ; "+" ; CC1=C(C#N)C(=C(C#N)C#N)OC1(C)c1ccccc1 ; CC1(c2ccccc2)OC(=C(C#N)C#N)C(C#N)=C1/C=C/C1=C(Cl)C(=C/C=C2/C(C#N)=C([C-](C#N)C#N)OC2(C)c2ccccc2)/CCC1 ; [Na+]]

The dialdehyde 3 and the tricyanofuran 2b were added into acetic anhydride supplemented with sodium acetate such that the molar ratio among the dialdehyde 3, the tricyanofuran 2b, and the sodium acetate was 1.0:2.1:2.2. The mixture was reacted by stirring at 120° C. for 4 hours to synthesize cyanine compound 4b. The obtained cyanine compound 4b was purified, and its yield was measured and was consequently 38%. Results of NMR measurement thereof are shown below.

¹H NMR (Acetone-d₆) δ 1.65-1.71 (m, 2H, —CH₂CH₂CH₂—), 2.08-2.09 (m, 6H, —CH₃ X 2), 2.35-2.46 (m, 4H, —CH₂CH₂CH₂—), 6.05 (d, J=14.2 Hz, 1H, vinyl H), 6.07 (d, J=14.2 Hz, 1H, vinyl H), 7.38-7.47 (m, 6H, aryl H), 7.50-7.52 (m, 4H, aryl H), 7.60 (d, J=14.2 Hz, 1H, vinyl H), 7.65 (d, J=14.2 Hz, 1H, vinyl H).

### Reference Example 2

Cyanine compound 5b was synthesized in accordance with the following scheme.

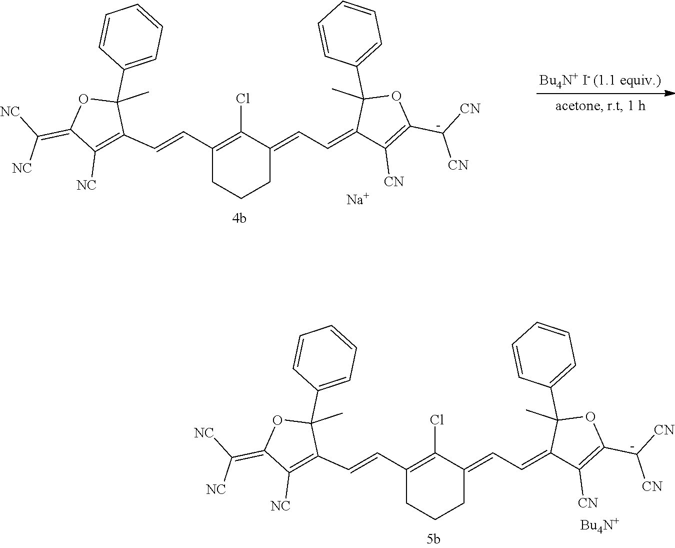

The cyanine compound 4b obtained in Reference Example 1 was added into an acetone solution supplemented with tetrabutylammonium iodide such that the molar ratio between the cyanine compound 4b and the tetrabutylammonium iodide was 1.0:1.1. The mixture was reacted by stirring at room temperature for 1 hour to synthesize cyanine compound 5b. The obtained cyanine compound 5b was purified, and its yield was measured and was consequently 65%. Results of NMR measurement thereof are shown below.

¹H NMR (Acetone-d₆) δ 0.98 (t. J=7.3 Hz, 12H, —CH₂CH₂CH₂CH₃ X 4), 1.39-1.48 (m, 8H, —CH₂CH₂CH₂CH₃ X 4), 1.67-1.73 (m, 2H, —CH₂CH₂CH₂—), 1.80-1.87 (m, 8H, —CH₂CH₂CH₂CH₃ X 4), 2.10 (m, 6H, —CH₃ X 2), 2.38-2.48 (m, 4H, —CH₂CH₂CH₂—), 3.43-3.47 (m, 8H, —CH₂CH₂CH₂CH₃ X 4), 6.05 (d, J=14.2 Hz, 1H, vinyl H), 6.07 (d, J=14.2 Hz, 1H, vinyl H), 7.39-7.48 (m, 6H, aryl H), 7.51-7.53 (m, 4H, aryl H), 7.53 (d, J=14.2 Hz, 1H, vinyl H), 7.66 (d, J=14.2 Hz, 1H, vinyl H).

### Comparative Example 1

A compound represented by the following formula was synthesized by a method known in the art.

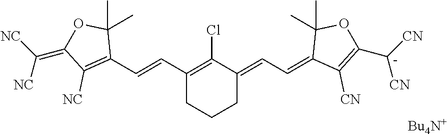

The local maximum absorption in a dichloromethane solution (1×10⁻⁶ M) of the compound obtained in each of Example 2, Reference Example 2 and Comparative Example 1, and the transmittance thereof in each wavelength region were measured using a spectrophotometer manufactured by Hitachi High-Tech Corp. (product name: U-4100). As one example thereof, the absorption spectra of the compound of Example 2 are shown in FIG. 2. In the obtained absorption spectra, the local maximum absorption wavelength was 934 nm in Example 2, 920 nm in Reference Example 2, and 906 nm in Comparative Example 1.

<Light Resistance>

In a thermostat bath of 25° C., a dehydrated dichloromethane solution (1×10⁻⁶ M) of the compound obtained in each of Example 2, Reference Example 2 and Comparative Example 1 was continuously irradiated with a white LED light (L-711) to examine the residual ratio of the compound in the solution (the concentration of the compound in the solution immediately before irradiation is defined as 100%). Thirteen days after the start of irradiation, the residual ratio of the compound of Example 2 was 69%, whereas the residual ratio of the compound of Reference Example 2 was 45%. The residual ratio of the compound of Comparative Example 1 fell below the detection limit 12 days after the start of irradiation.

<Heat Resistance>

The decomposition temperature of the compound obtained in each of Example 2, Reference Example 2 and Comparative Example 1 was measured by TG-DTA (apparatus name: EXSTAR-6000 TG/DTA 6300, manufactured by Seiko Instruments Inc.). The sample used in measurement was subjected to heat drying treatment under reduced pressure (80° C., 3×10² Pa, overnight) in advance. In the measurement, the temperature was elevated from 30° C. to 400° C. in a nitrogen atmosphere, and the temperature at which the weight was decreased by 2% was measured. The temperature at which the weight of the compound of Example 2 was decreased by 2% was approximately 207° C., whereas the temperature at which the weight of the compound of Reference Example 2 was decreased by 2% was approximately 200° C. and the temperature at which the weight of the compound of Comparative Example 1 was decreased by 2% was approximately 198° C.

The present application is based on the Japanese patent application filed on Sep. 25, 2020 (Japanese Patent Application No. 2020-161368), the contents of which are incorporated herein by reference.

### INDUSTRIAL APPLICABILITY

The cyanine compound of the present invention has absorption of near-infrared light of more than 800 nm and has no or minimum sensitivity to visible light. The cyanine compound of the present invention is also excellent in durability performance such as light resistance and heat resistance. Hence, the cyanine compound of the present invention can be used as a material for a photoelectric conversion element that has transparency, generates a charge in response to near-infrared light, and is excellent in durability. Thus, the cyanine compound and the photoelectric conversion element of the present invention have industrial applicability in fields that require these characteristics. Specifically, the cyanine compound and the photoelectric conversion element of the present invention have industrial applicability as a solid image sensor in, for example, image sensors for security cameras, in-car cameras, cameras for uninhabited air vehicles, agricultural cameras, industrial cameras, medical cameras such as endoscopic cameras, cameras for game machines, digital still cameras, digital video cameras, cameras for mobile phones, and cameras for the other mobile instruments; image scanning elements for facsimiles, scanners and copying machines; and photosensors for biosensors and chemical sensors. Also, the cyanine compound and the photoelectric conversion element of the present invention have industrial applicability as a display in, for example, television monitors, touch monitors, digital signages, wearable displays, electronic papers, and head-up displays for mobility application. The cyanine compound of the present invention also has industrial application, for example, as a material for optical information recording media, flash fusing photosensitive materials, thermal shielding films, infrared cut filters, anti-counterfeit ink, or as a preform heating auxiliary agent intended for plastic bottles, in addition to those mentioned above.

### REFERENCE SIGNS LIST

100 . . . infrared photoelectric conversion unit, 110 . . . organic infrared photoelectric conversion film, 120 . . . hole transport layer, 130 . . . electron transport layer, 140, 150 . . . electrode.

1. A cyanine compound being bound counterions consisting of an anion and a cation, wherein the anion is represented by the following formula (I-1): 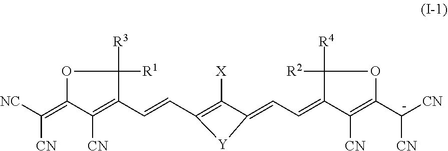
wherein R¹ and R² each independently represent a hydrogen atom or a monovalent organic group; R³ and R⁴ each independently represent a monovalent group represented by the following formula (I-1-1); X represents a hydrogen atom, a halogen atom or a monovalent organic group; and Y represents a divalent group represented by the following formula (I-1-2) or (I-1-3): 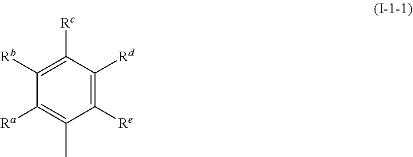
wherein R^{a}, R^{b}, R^{c}, R^{d} and R^{e} each independently represent a hydrogen atom, a monovalent hydrocarbon group or a monovalent electron-withdrawing group; one or more of R^{a}, R^{b}, R^{c}, R^{d} and R^{e} represent the monovalent electron-withdrawing group; and when one of R^{a}, R^{b}, R^{e}, R^{d} and R^{e} is a halogen atom, one or more of the other moieties of R^{a}, R^{b}, R^{e}, R^{d} and R^{e} represent the monovalent hydrocarbon group or the monovalent electron-withdrawing group, 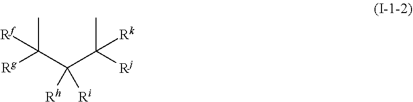
wherein R^{f}, R^{g}, R^{h}, and R^{k} each independently represent a hydrogen atom, or a monovalent hydrocarbon group optionally having oxygen atom(s), nitrogen atom(s) or sulfur atom(s), and 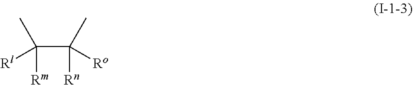
wherein R^{l}, R^{m}, Rⁿ and R^{o} each independently represent a hydrogen atom, or a monovalent hydrocarbon group optionally having oxygen atom(s), nitrogen atom(s) or sulfur atom(s).

2. The cyanine compound according to claim 1, wherein the cation comprises one or more selected from the group consisting of an alkali metal cation, an alkaline earth metal cation, an ammonium cation, a sulfonium cation, a phosphonium cation and cationic cyanine.

3. The cyanine compound according to claim 2, wherein the cation comprises one or more selected from the group consisting of an alkali metal cation, an ammonium cation and cationic cyanine.

4. The cyanine compound according to claim 3, wherein the cationic cyanine is a cation represented by the following formula (I-2-1), (I-2-2), (I-2-3) or (I-2-4): 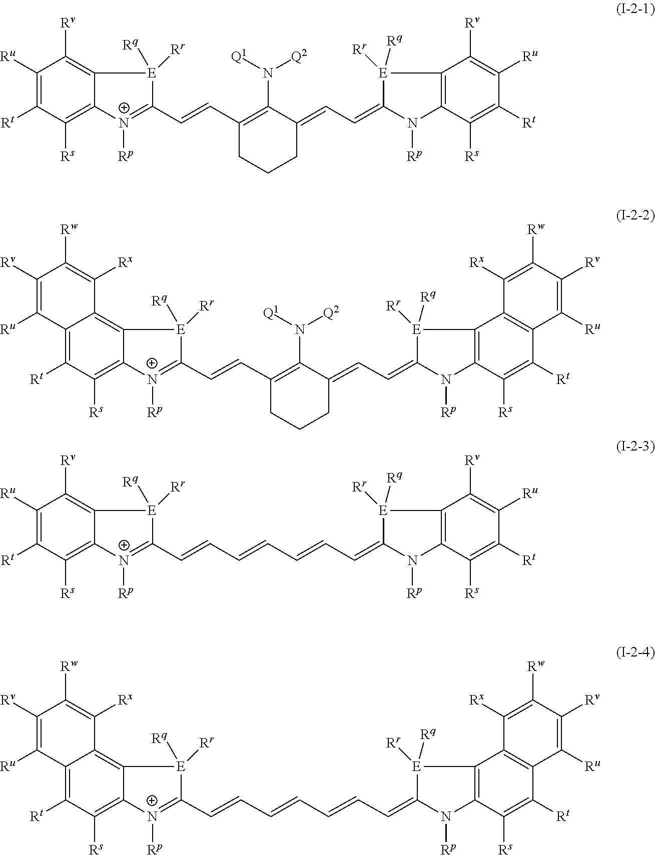
wherein
each E independently represents a carbon atom, a nitrogen atom, an oxygen atom or a sulfur atom;
R^{p}, R^{q}, R^{r}, R^{s}, R^{t}, R^{u}, R^{v}, R^{w} and R^{x} each independently represent a hydrogen atom, a halogen atom, a hydroxy group, a carboxy group, a nitro group, an amino group, an amide group, an imide group, a cyano group, a silyl group, -L¹, —S-L², —SS-L², —SO₂-L³, —N═N-L⁴, or one or more groups selected from the group consisting of groups represented by the following formulas (A), (B), (C), (D), (E), (F), (G) and (H) having one or more combinations of R^{q} and R^{r}, R^{s} and R^{t}, R^{t} and R^{u}, R^{u} and R^{v}, R^{v} and R^{w}, and R^{w} and R^{x} bonded to each other, wherein
the amino group, the amide group, the imide group and the silyl group can be each further substituted by one or more groups L selected from the group consisting of a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms and a monovalent heterocyclic group having 3 to 14 carbon atoms;
each of the L¹ and the L⁴ is a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L;
the L² is a hydrogen atom, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L; and
the L³ is a hydroxy group, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L;
Q¹ represents an acetyl group; and Q² represents a structure represented by the following formula (q1), (q2) or (q3): 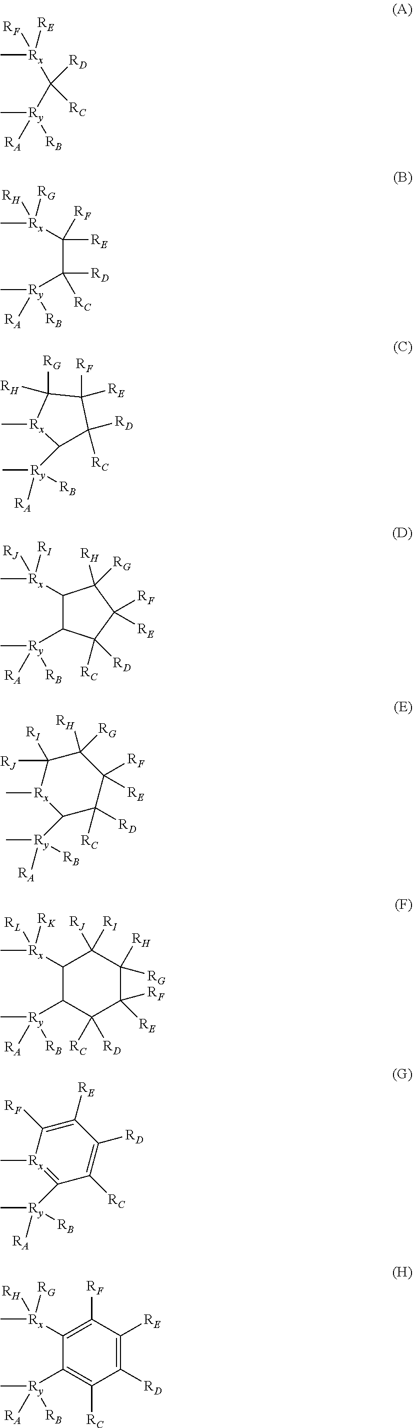
wherein the combination of R^{x} and R^{y} is a combination of R^{q} and R^{r}, R^{s} and R^{t}, R^{t} and R^{u}, R^{u} and R^{v}, R^{v} and R^{w}, or R^{w} and R^{x};
R_{A}, R_{B}, R_{C}, R_{D}, R_{E}, R_{F}, R_{G}, R_{H}, R_{I}, R_{J}, R_{K} and R_{L} each independently represent a hydrogen atom, a halogen atom, a hydroxy group, a carboxy group, a nitro group, an amino group, an amide group, an imide group, a cyano group, a silyl group, -L¹, —S-L², —SS-L², —SO₂-L³ or —N═N-L⁴, wherein L¹, L², L³ and L⁴ are as defined in L¹, L², L³ and L⁴ in the formulas (I-2-1) and (I-2-2), and the amino group, the amide group, the imide group and the silyl group can be substituted by the group(s) L,
—CₘHₘ₊₁ (q1)
—CₐHₐ₊₁—OC_{b}H_{b+1} (q2)
wherein m in the formula (q1) represents an integer of 1 to 5, and a and b in the formula (q2) each represent an integer of 1 to 5, and 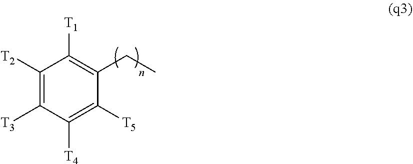
wherein n represents an integer of 1 to 5; T₁, T₂, T₃, T₄ and T₅ each independently represent a hydrogen atom or —OCₚHₚ₊₁; and p represents an integer of 1 to 5.

5. The cyanine compound according to claim 1, wherein the monovalent organic group represented by each of the R¹ and the R² is a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by a monovalent hydrocarbon group or a monovalent electron-withdrawing group.

6. The cyanine compound according to claim 5, wherein the R¹ and the R² are each independently a hydrogen atom, a monovalent aliphatic hydrocarbon group having 1 to 3 carbon atoms or a monovalent group represented by the formula (I-1-1).

7. The cyanine compound according to claim 1, wherein
the monovalent organic group represented by the X represents a hydroxy group, a carboxy group, a nitro group, an amino group, an amide group, an imide group, a cyano group, a silyl group, -L¹, —S-L², —SS-L³, —SO₂-L³, or —N═N-L⁴, wherein
the amino group, the amide group, the imide group and the silyl group can be each further substituted by one or more groups L selected from the group consisting of a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms and a monovalent heterocyclic group having 3 to 14 carbon atoms;
each of the L¹ and the L⁴ is a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L;
the L² is a hydrogen atom, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L; and
the L³ is a hydroxy group, or a monovalent aliphatic hydrocarbon group having 1 to 12 carbon atoms, a monovalent halogen-substituted alkyl group having 1 to 12 carbon atoms, a monovalent alicyclic hydrocarbon group having 3 to 14 carbon atoms, a monovalent aromatic hydrocarbon group having 6 to 14 carbon atoms or a heterocyclic group having 3 to 14 carbon atoms, each of which can be further substituted by the group(s) L.

8. The cyanine compound according to claim 7, wherein the X is a halogen atom.

9. The cyanine compound according to claim 1, wherein the monovalent hydrocarbon group represented by each of the R^{a}, the R^{b}, the R^{c}, the R^{d} and the R^{e} is a monovalent aliphatic hydrocarbon group having 1 to 12 carbon atoms, a monovalent alicyclic hydrocarbon group having 3 to 14 carbon atoms or a monovalent aromatic hydrocarbon group having 6 to 14 carbon atoms, each of which can be further substituted by one or more groups selected from the group consisting of a monovalent aliphatic hydrocarbon group having 1 to 12 carbon atoms, a monovalent alicyclic hydrocarbon group having 3 to 14 carbon atoms and a monovalent aromatic hydrocarbon group having 6 to 14 carbon atoms.

10. The cyanine compound according to claim 1, wherein the monovalent electron-withdrawing group represented by each of the R^{a}, the R^{b}, the R^{c}, the R^{d} and the R^{e} is a halogen atom, a carboxy group, a nitro group, a cyano group, a group represented by —COR, a group represented by —CONR₂, a group represented by —SO₂R or a group represented by —SO₃R, wherein the R is as defined in the monovalent hydrocarbon group or a hydrogen atom.

11. The cyanine compound according to claim 10, wherein the R^{a}, the R^{b}, the R^{c}, the R^{d} and the R^{e} each independently represent a hydrogen atom or a halogen atom, and two or more of R^{a}, R^{b}, R^{c}, R^{d} and R^{e} are halogen atoms.

12. The cyanine compound according to claim 1, wherein the monovalent hydrocarbon group optionally having oxygen atom(s), nitrogen atom(s) or sulfur atom(s), represented by each of the R^{f}, the R^{g}, the R^{h}, the Rⁱ, the R^{j}, the R^{k}, the R^{l}, the R^{m}, the R^{h} and the R^{o} is a monovalent aliphatic hydrocarbon group having 1 to 12 carbon atoms, a monovalent alicyclic hydrocarbon group having 3 to 14 carbon atoms or a monovalent aromatic hydrocarbon group having 6 to 14 carbon atoms, each of which can be further substituted by one or more groups selected from the group consisting of a monovalent aliphatic hydrocarbon group having 1 to 12 carbon atoms, a monovalent alicyclic hydrocarbon group having 3 to 14 carbon atoms and a monovalent aromatic hydrocarbon group having 6 to 14 carbon atoms, each of which optionally has oxygen atom(s), nitrogen atom(s) or sulfur atom(s).

13. The cyanine compound according to claim 12, wherein R^{f}, R^{g}, R^{h}, Rⁱ, R^{j}, R^{k}, R^{l}, R^{m}, Rⁿ and R^{o} each independently represent a hydrogen atom, or a monovalent aliphatic hydrocarbon group having 1 to 12 carbon atoms.

14. A photoelectric conversion element comprising an infrared photoelectric conversion unit comprising a pair of electrodes and an organic infrared photoelectric conversion film disposed between the pair of electrodes, wherein
the organic infrared photoelectric conversion film comprises the cyanine compound according to claim 1.

15. The photoelectric conversion element according to claim 14, wherein the organic infrared photoelectric conversion film comprises an organic n-type semiconductor and/or an organic p-type semiconductor.

16. The photoelectric conversion element according to claim 14, wherein the infrared photoelectric conversion unit comprises one or more selected from the group consisting of a hole transport layer, an electron transport layer, a hole blocking layer, and an electron blocking layer between the electrode and the organic infrared photoelectric conversion film.

17. The photoelectric conversion element according to claim 14, wherein in the infrared photoelectric conversion unit, a local maximum absorption wavelength and a maximum absorption wavelength of optical absorption spectra in the infrared region are 800 nm or more and 2500 nm or less.

18. The photoelectric conversion element according to claim 14, wherein the photoelectric conversion element further comprises a visible photoelectric conversion unit having sensitivity to a light in the visible region.
